(19) United States
(12) Patent Application Publication
(10) Pub. No.: US 2015/0134022 A1
(43) Pub. Date: May 14, 2015

(54) Cell electric stimulator with separate electrodes for electrical field shaping and for stimulation

(71) Applicants: Chong Il Lee, Stanton, CA (US); Sergio Lara Pereira Monteiro, Los Angeles, CA (US)

(72) Inventors: Chong Il Lee, Stanton, CA (US); Sergio Lara Pereira Monteiro, Los Angeles, CA (US)

(21) Appl. No.: 14/495,871

(22) Filed: Sep. 24, 2014

Related U.S. Application Data
(60) Provisional application No. 61/881,997, filed on Sep. 25, 2013.
(60) Provisional application No. 62/027,116, filed on Jul. 21, 2014.

(51) Int. Cl. A61N 1/36; A61N 1/362; A61N 1/05; A61N 1/372

(52) U.S. Cl. CPC A61N 1/36185; A61N 1/37223; A61N 1/362; A61N 1/05; A61N 1/0529
USPC 607/9; 607/2; 607/60; 607/116

(57) ABSTRACT

An electric stimulator for heart, brain, organs and general cells with a possibly random shape and position of electrodes which enhances its performance for breaking the symmetry. Two types of electrodes are introduced: type-1, or active electrodes are similar to prior art, while type-2, or passive electrodes have not been used in this context. Passive electrodes are electrically insulated, being unable to inject current in the surrounding medium, but they are capable of shaping the electric field, which has consequence on the path of the stimulating currents injected by type-1 electrodes. The invention also discloses a supercapacitor-type passive electrode of type-2, which maximizes the stored charge on the electrode—therefore increasing the electric field magnitude created by it.

### CROSS-REFERENCE TO RELATED APPLICATIONS

This application claims priority to U.S. provisional patent application No. 61/881,997 dated 25 Sep. 2013, entitled “Cell electric stimulator with electrodes for electrical field shaping and separate electrodes for stimulation” and U.S. provisional patent application No. 62/027,116 dated 21 Jul. 2014, entitled “Cell electric stimulator with electrodes for electrical field shaping and separate electrodes for stimulation”, with the same inventors as this patent application.

This application is related to U.S. Provisional Patent Application No. 61/486,179 dated 13 May 2011, entitled “Cell electric stimulator with randomized spatial distribution of electrodes for both current injection and for field shaping”, later regular U.S. patent application Ser. No. 13/470,275, application date 12 May 2012, published with number US-2012-0289823 A1 on 15 Nov. 2012, currently allowed, and also related to European patent application number EP 2012 0167688.6 application date 11 May 2012, publication number EP2522389 A3 on Aug. 27 2014. We incorporate here, by reference, the full text, figures and claims of all these provisional and regular applications.

### FEDERALLY SPONSORED RESEARCH

Not applicable

### SEQUENCE LISTING OR PROGRAM

Not applicable

### BACKGROUND OF THE INVENTION

1. Field of Invention

This invention relates to electrical stimulation of cells in animals and other living forms, particularly to electrical stimulation of heart cells, including heart muscles associated with heart muscle contraction and with the His bundle, the left and right bundles, the Purkinje and similar fibers. The invention is applicable to artificial heart pacemakers. More precisely, the invention relates to causing an efficient contraction sequence of the heart muscle in order to maximize the volume of blood pumped per unit of energy spent by the heart. It also relates to the field of electrical stimulation of the cochlea, as in cochlear implants. It also relates to the field of electrical stimulation of neurons as in brain and peripheral neurons. Brain neurons are stimulated both for clinical objectives, as in Parkinson's disease control, and in animal research as well, in which case neurons are stimulated to observe the consequences of the stimulation. Other neurons are stimulated to block pain sensation on its way to the brain or other information transmitting function. An example of the latter is the information to the brain about the blood pressure in arteries and veins, as discussed in Dennis T. T. Plachta et al. [Plachta 2014]. It also relates to the field of electrical stimulation of organs, as stomach, etc.

2. Discussion of Existing Devices and Known Pertinent Facts

The phenomenon of muscle contraction and its electrical nature was first observed in the waning years of the 1700s by the great Italian Luigi Galvani (born 1737, dec. 1798), from Bologna, who noticed that a frog's leg contracted when subjected to an electric current. Today it is known that all our muscles, from a blinking eye to a walking leg and fingers pressing the keyboard of a computer to write the background section of a patent application work on the same principles observed by Galvani—including out heart. The heart contracts as response to an electric pulse, which is injected on it at the required frequency, which varies according to the person's activity and state of excitation.

Broken to their building blocks, existing heart pacemakers are an electrode, which is a fancy name for a tip of exposed metal, an electric battery and a controlling electronic circuit capable of generating pulses at the heart beat frequency of approximately 70 per minute, or a little less than one second each. The electrode is anchored in the heart, the battery and the controlling electronic circuit are located in a sealed box, usually just below the skin, in the chest of abdomen, with a connecting wire from the battery/controlling electronics to the electrode in the heart. The battery/controlling electronics are located in a sealed box 110 is of the approximate size as an ordinary cell phone, but with a much simpler electronics controlling unit, though, for some mysterious reason that escapes me the heart device costs one hundred times more than a cell phone. As it is known to the persons familiar with the electronics fabrication units, the cost disparity is not due to the need to keep the heart pacemaker clean of germs, because the electronics fabrication units are far cleaner than any surgery room. The electronic circuit is capable of creating an electric pulse at some periodicity, and capable of injecting a certain current in the region surrounding the electrode in the heart. The electrode itself is implanted in the heart, usually via a simple procedure involving inserting a wire with the electrode at its tip from a vein just below the clavicle (the sub-clavian vein), or some other convenient blood vessel, feeding the wire in while watching on an X-ray machine until the electrode reaches the heart, then anchoring the electrode into the inner wall of the heart. Variations of this basic design involve pacing-on-demand, which means pacing only when the natural mechanism fails, or a double or even triple electrode, and many other bells and whistles.

It is crucial here to keep in mind the difference between the electric current in metals (as in wires) and the electric current through the cells of an animal, as a human. The electric current in metals propagates by the motion of electrons, which are light particles moving through a mostly unopposed medium of the metal known as the conduction band; the electric current in wires go around the equator 5 times in a second (⅔ of the speed of light). The electric current in animal cells propagates by the motion of heavy ions (usually K or Ca) in a difficult path to negotiate, suffering many collisions, besides dragging charges of the opposite polarity inside the cells, which increase their effective masses; they go the 10 cm length of the heart in 1 second—2 billion times slower than electrons in wires. It is the slow speed (and longer propagation time) that allows for the manipulation of the electric charges—in time and space, as done by our invention, as described in the sequel. The reader is requested to keep this in mind, that the electric pulse propagation within the heart muscle is extremely slow as far as electric phenomena go.

Several malfunctions are possible to occur that hinder the proper functioning of the heart. Some are of a mechanical nature, a subject not bearing on our invention, while some are of an electrical nature, which is the focus of our invention, as described later on: our invention is an inventive method and means to cause a better propagation of the electric pulse that causes the heart to contract—and consequently, our invention is an inventive method and system to cause a better heart pumping. Better is here used in the sense of pumping more blood for a fixed amount of energy spent for the activity.

There are a wealth of books on the subject of heart contraction. A simple book is Thaler (2003), where the reader with a non-medical background can get more detailed information. In short, most muscles capable of contracting are made of such cells that under normal conditions they have an excess of negative ions inside their cellular walls, which in turn causes an excess of positive ions just outside their cellular walls, attracted there by ordinary electrostatic attraction caused by negative ions inside the cell. When in this condition, its normal condition, the cell is said to be polarized (medical parlance). If an electric charge is introduced at some point in the muscle, this charge causes a propagating chain of motion of charges, similar to a falling domino sequence, which its associated propagating contraction sequence. This is the mechanism behind the blinking of our eyes, behind our walking, behind the sideways shaking head and the smile of pity of a physician reading this simplistic physicist's view of body cells—and also behind the heart contraction. The heart contraction is an electric driven phenomenon, caused by the injection of the appropriate electrical pulse in the heart muscle at the top of the right atrium and the heart pacemaker is simply an electrode capable of injecting an electric charge at some desired positions in the heart muscle. This will be described in the sequel, and our invention bears on a twist on the man-made mechanism (artificial heart pacemaker) designed to cause an optimized heart pumping contraction sequence. Our invention improves on the propagation of the artificial electric pulse that causes a heart contraction (and consequent blood pumping).

As a last preparatory information we want to clarify that the heart pumping mechanism is a modification of a class of pumps called peristaltic pump, which causes the motion of the fluid, or pumping, with a progressive forward squeezing of the container, which forces the fluid forward. If the reader is unfamiliar with the mechanism of peristaltic pumping, we recommend that she/he acquaints her/himself with the method, perhaps observing the animation in the wikipedia article on peristaltic pump, or any similar source. The inventors suspect that the cardiologists are not generally aware of the progressive forward squeezing of the heart, and that when the cardiologists states that the heart contracts sequentially many only means that the atria (top part of the heart) contracts before the ventricles (bottom part of the heart), as opposed to the sequential forwarding squeezing of each cavity. This is partly because the heart moves up and down and also sideways while twisting widely through each cycle, which hides the observation, but above all because the progressive squeezing is imperfectly made. So, repeating, within each of the two cycles the actual contractions are sequential in the sense that the muscles start contracting at one extremity (say, the top of the atrium) then sequentially contracting down, toward the exit valve at the bottom—as a thoughtful person squeezes the toothpaste tube. This latter sequential contraction is the one the inventors want to bring forth—and a sequence that, alas, many a cardiologist will deny.

One example of a peristaltic pump is the caw milking. Unfortunately very few people have ever milked a caw, including the inventors, so this is a gedanken experiment (one of us checked for its accuracy with a farmer, having obtained confirmation of its accuracy). The milker holds the caw's tit between her/his four fingers with the thumb up, near the caw's udder, (pointing, middle, annular and little fingers) then progressively squeezes the caw's tit between its pointing finger and the palm of her/his hand, then press the middle finger while holding the pointing finger closed, to prevent back motion of the milk, squeezing the stored liquid further down from the tit, then the annular than the little finger, all along keeping the previously closed fingers closed to prevent backward motion of the milk. Having pressed the small finger, all the can be squeezed is out, the hand is opened to allow more milk to enter the tit and the process is repeated.

Another example, this one only partly representative of a peristaltic pump, is the toothpaste tube. This example is easier to understand because all readers of this document brush their teeth regularly—or so we hope, so this is more than a gedanken experiment! For the best effect, the toothpaste tube should be squeezed from the back on forward. Accordingly, a thoughtful person squeezes the tube from the back end advancing forward as the tube empties, perhaps even rolling the back on itself if the tube is stiff enough to be compatible with this, to close the back volume, forestalling backwards motion of the toothpaste. Scattered minded people squeeze the paste tube from the middle, a practice that drives thoughtful people crazy and have, the inventors suspect, caused many divorces. Squeezing the toothpaste tube from the middle causes a most inefficient toothpaste ejection (pumping).

The reader is requested to keep this fact in mind as she/he reads the explanation of our invention, that the hearts functions with a progressive squeezing of its chambers, akin to the milking of a caw, or to the squeezing of the toothpaste tube in such a way as to preserve his/her marriage. The perfect heart is equivalent of a toothpaste tube squeezed from the back. For such forward squeezing the heart requires an appropriate electric pulse propagation along the 3-D heart muscle, to keep it contracting in the appropriate sequence, and conversely, deviation from the ideal electrical pulse propagation causes a non-ideal heart squeezing sequence, which is mimicked by a toothpaste tube being squeezed at the middle, causing a non-efficient pumping.

In short, most of the heart cells are part of the miocardium, which is a variety of a large group of other cells which are capable of contracting when subjected to the mechanism just described of depolarization. FIG. 1 shows the major part of a human heart. Note the four main chambers: right atrium RA, right ventricle RV, left atrium LA, left ventricle LV, and some of the main parts of the heart: sinus node or sinus-atrial node (SN or SAN), atrial ventricular node (AV or AVN), both of which are the starting points for the electrical pulses, the His bundle (HB), the right and left bundles (RB and LB) and the Purkinje fibers (PF), which are the “fast wires” responsible for the fast propagation of the electric pulse from the AVN node to the bottom of the ventricles, and the two inter chamber one-way valves: the tricuspid valve (on the right side) and the mitral valve (on the left side). The pumping sequence consists of blood entering the heart at the top of the atrium (which is the upper chamber) during the relaxation cycle of the atrium. This is then followed by the introduction of an electric pulse starting at a small group of cells known as the sino-atrial node (SA node or SAN), from where the electric charges propagate downwards, causing a sequential downward pumping that sequentially squeezes of the atrium downwards, forcing the blood into the lower ventricle. This is the P wave in an electrocardiogram, or EKG. Then there is a problem because the exit of the lower part of the heart, the ventricle, is at its top, next to its entrance port, so, if the squeezing continued downward there would be no place for the blood to go (no exit port at the bottom of the ventricle!). This problem is solved with the interruption of the downward propagating electric pulse at the intersection of these two chambers and a re-emission of another pulse through fast channels known as His bundle HB, left and right bundles, LB and RB, and finally the Purkinje fibers PF, which release the electrical pulse at the base of the ventricles, RV and LV, which then begin squeezing from the bottom to top, squeezing the blood upwards towards the exit port (the pulmonary vein at the right ventricle and the aorta at the left ventricle). This is the QRS complex on the electrocardiogram. After the ventricles complete their pumping a new cycle start with another contraction of the atrium, or upper chamber.

The original artificial heart pacemakers simply injected an electric pulse near the sino-atrial node (SAN) at the top of the right atrium, and later versions injected two or even three separate pulses in two or three different parts of the hearts, with the appropriate time delays, which correspond to the elapsed time for the natural pulse to be at that place for a good contraction sequence. None of them, though, even attempted to control the path and the speed of the injected current once it is injected artificially—which is the object of our invention. In other words, our invention improves on the electrical propagation features of the electric pulse created by the artificial heart pacemakers, and in doing so it improves the squeezing sequence of the heart, which in turn improves the pumping efficiency. It is to be remembered that because the heart is a variation of a peristaltic pump, the pumping sequence is of fundamental importance for an efficient pumping (the inventors hope that the reader did indeed go see the animation in Wikipedia or elsewhere).

Originally heart pacemakers were simply an exposed wire wire tip, the wire connected to a battery and electronics circuitry to create pulses of appropriate frequency, duty cycle and amplitude. The original implant was made with an open chest surgery, but this was quickly supplanted by a less invasive and much less traumatic technique, with which an incision was made on some vein at the chest (usually the subclavian vein, SCV, FIG. 2, on the upper chest), where a wire was inserted, which had some sort of anchoring ending at its distal extremity, then this wire was fed into the blood vessel until its distal extremity reached the upper right heart chamber, from the inside (the right atrium), where the wire tip was anchored on the inner part of the heart, near the natural starting point of the electrical pulse that causes the heart to beat, know as the sino-atrial node (SA node or SAN). During this process the patient lays in an X-ray imaging system and the surgeon can observe the advancement of the wire down the vein on an X-ray monitor. The proximal end of the wire was then connected to a battery BAT1 and electronics box 110 which was implanted in the chest, in some convenient location. From the wire tip anchored at the distal end, a current emanated, which then propagated through the heart muscle, causing the muscle to contract as the current proceeded along it, hopefully similarly to the naturally occurring electric pulse. It is crucial here to remember that this muscle contraction occurs because of the forward propagation of the injected electric charge, and consequently, it is the electric current propagation time and pathway that determines the heart contraction sequence, in time and space—because the muscle cells contract as a consequence of the electric charge arriving to its location. The sequence of muscle contraction is crucial for an efficient heart functioning, because the heart must start squeezing from its furthest end, away from the discharge exit area, most away from the exit port, continuously squeezing its walls towards the exit port. The heart does not contracts as a person squeezes a tennis ball for exercise, but rather, the heart squeezes sequentially pushing the blood forward, towards the exit port. The reader can here recall the caw milking and the toothpaste tube described above.

Most people get astonished when they learn that the heart of an athlete at her/his peak pumps only 75% of the blood volume inside it (I was!), that a health young adult pumps only 70% of the blood inside it, then goes down from there until when the heart of an older, inactive person starts pumping less than 50% it becomes time for some intervention. The heart operates with a rather low efficiency! So much for intelligent design. Intelligent it was not.

Over the more than 50 years of heart pacemaking, many types of electrode tips have been developed. Some of the electrode tips possessed some degree of symmetry, some not. Whether the tip electrode had or did not have symmetry, this property was initially transferred to the current injected into the heart muscle—but only initially. The heart, on the other hand, is asymmetric, particularly from the point of view of the point where the stimulating electrode is anchored in the heart, which often is near the sino-atrial node SAN, or at the top of the right atrium. It follows that the current that is injected by existing heart pacemakers can hardly be expected to follow well the contour of the heart muscle, much less with a correct timing for a proper squeezing sequence, causing a less than ideal contracting sequence. Other anchoring positions for the electrode are also used, and multiple electrodes as well, which may stimulate the atrium and the ventricle independently, and the multiple electrodes heart pacemakers, also known as Cardiac Resynchronization Therapy, is a step in the right direction of controlling the timing of the start of the upper contraction (the atria) and the start of the lower contraction (the ventricles), but still an insufficient step for failure to control the electric charge wave continuously, as it travel through the heart muscle. Cardiac Resynchronization Therapy controls the starting time of the contractions but not the progression in time of them. What is needed is a device which is capable of continuously controlling the path and speed of the current as it travels through the heart muscle.

At this point we ask the medical people, particularly the cardiologists and the electrophysiologists to ponder on the need to control the contraction sequence further than only its initial timing, as done by Cardiac Resynchronization Therapy, controlling the progression of the contraction too, all along the heart wall. Current devices cannot control the path and timing of the injected current because the electrodes are on for a very short time, of the order of 1% of the total time. Electrophysiologists ought to be emotionally prepared to accept this new trick of controlling the path and time of the injected current.

There exist one exception to the adjustment of the path for the propagating current in the heart, one that shows that the cardiologists are aware of the problem but have not solved it yet—not for the heart pacemakers. This exception of adjustment is the surgery known as catheter ablation, which consists of selectively destroying selected groups of heart cells with the objective of redirect the path for the propagating currents. Current version of wikipedia (on 21 Sep. 2014) states that: “Catheter ablation is an invasive procedure used to remove or terminate a faulty electrical pathway from sections of the hearts of those who are prone to developing cardiac arrhythmias such as atrial fibrillation, atrial flutter, supraventricular tachycardias (SVT) and Wolff-Parkinson-White syndrome.” It is worth to bring this to the attention of the readers because it shows that the problem we solve with our invention is an old known problem which have never been solved, even if so many competent and creative persons have tried to solve it.

As for electrode symmetry and current spread, in the former case, the tip symmetry had consequences on the current distribution in the heart muscle, because, at least initially, it imparted to the injected current the same symmetry as the symmetry of the causative agent, that is the same symmetry of the electrode. But the heart is not symmetric, so this initial symmetry was undesirable in principle. Therefore this symmetry, was not the ideal initiator of the heart contraction sequence. The message here is that the trajectory of current injection has not been controlled by existing devices, which is a major problem as acknowledged by cardiologists working in the field of electrophysiology. This lack of control of the current distribution, as it propagated through the heart muscle, plagued all the earlier types of heart pacemakers, and still does in existing pacemakers. Throughout the years, many variations were introduced in the electrodes, as the shape of the wire tip, which served to anchor it in place, but these changes were largely for mechanical reasons, as to provide a more secure anchoring of the electrode on the heart muscle, or to minimize physical damage to the heart tissues, etc. Changes have also occurred on the method of introducing it in the heart, but most of these were changes to solve other problems, not to induce a good squeezing sequence of the heart muscle. Consequently, the uncontrolled propagation of the electric current from the initiating tip has been a constant problem on all prior and existing heart pacemakers. Attempts to improve the electric pulse propagation include the use of multiple wire tips, which injected current not only at different locations but also at different times, or with relative time delay between the stimulating places. Examples of such multiple site stimulation are atrial and ventricular stimulators, two tips, one at the atrium, another at the ventricle, which deliver a pulse with a time lag between them, corresponding to the time lag between atrial contraction and ventricular contraction. But these multiple stimulating tips are not designed to control the electric field—which determines the path of the injected electric current, which more or less follows the electric field lines because these are the force lines.

Such multiple electrodes, usually worked better than a single electrode. Of course!, after all, they are a step in the direction of controlling the charge motion along the cycle, as opposed to just injecting an electric charge one time only at the beginning of the heart beating cycle. Yet, this lack of optimization of the heart muscle contraction has been a major problem known to many of the electrophysiologists and cardiologists. This uncontrolled propagation was shared by most, if not all models of heart pacemaker electrodes in use today, in spite of the fact that the cardiologists are well aware that uncontrolled electric pulse propagation caused inefficient heart pumping. Cardiologists knew that they had to address the problem of electric pulse propagation through the heart, but they have so far not succeeded in this goal. It has been a known problem in heart pacemakers, yet and amazingly, a problem which has defied solution for decades.

It seems that all existing devices, used now or in the past, attempts to solve the problem of electric pulse propagation inside the heart muscle tissues with the use of multiple electrodes, while nobody succeeded to control the current propagation, in direction and magnitude, using one or multiple electrodes. Nor have existing devices made full use of multiple electrodes to more completely shape the electric field within the heart muscle—which is the same as the electrical current path, because the electric field lines are the same as the lines of force on the electric charges, or the lines that direct the motion of the electric charges, similar to the steering wheel on an automobile.

The final conclusion is that existing electrodes simply used an arbitrarily shaped stimulating electrode with no control on the current after it is injected in the organ (say, the heart). Our invention offers a method and a means to adjust the electric field, independently from the stimulating electrodes, to the best shape depending on the particular case, as needed, including controlling the motion after the initial short time when the stimulating electrodes are energized.

### Objects and Advantages

Accordingly, several objects and advantages of our invention are one or more of the following. A better squeezing sequence of the heart muscle, starting the muscle contraction from the distal end of the heart further away from the exit port, to the proximal end of the heart closer to the exit port, with view to achieve a more efficient pumping, when compared with existing artificial heart pacemakers which were designed with no view to optimize the squeezing sequence.

Another object and advantage of our invention is to offer the ability to control the path of the electric current in the heart so as to cause a higher pumping fraction, or the fraction of the blood which is actually pumped out of it, or out of each chamber, when compared with existing artificial pacemakers in use today.

Another object and advantage of our invention is to adjust the electric field over the heart muscle to take better advantage of the atrial ventricular node to cause a better squeezing sequence of the heart muscle when compared with artificial pacemakers in current use.

Another object and advantage of our invention is to adjust the electric field over the part of the heart muscle where the His bundle and the right and left bundles and the Purkinje fibers are, to control the propagation times of the electric current coming from the atrial-ventricular node to the bottom and sides of the ventricle, to cause a better squeezing sequence of the ventricles heart muscle when compared with artificial heart pacemakers in current use.

Another object and advantage of our invention are a better volumetric fit of the neural electrical stimulation to the optimal heart and/or other tissues target volume, when compared with currently used electrical stimulation devices.

Another object and advantage of our invention for brain neural stimulation is to better control the electric field around the supporting structure from where electrical stimulation is injected in the target volume of the brain when performing Deep Brain Stimulation, to cause that the electrical stimulation reaches a larger volume of the target volume while better avoiding stimulating other parts of the brain that are near but outside and beyond the target volume.

Another object and advantage of our invention is the possibility of time control of the motion of charges for stimulation sequences in neural stimulation, which is not achieved with currently used devices.

Another object and advantage of our invention is a better control of the shape of the volume which contains the neurons that receive electrical stimulation in brain stimulation, as in DBS (Deep Brain Stimulation).

Another object and advantage of our invention is a better control of the shape of the volume of neurons that receive electrical stimulation in neural stimulation, as for TENS (Transcutaneous Electrical Neural Stimulation) pain control.

Another object and advantage of our invention is a better control of the shape of the superficial distribution of neurons as for pain control in TENS (Transcutaneous Electrical Neural Stimulation) devices.

Another object and advantage of our invention is a better control and shape of the mostly planar electrical stimulation of neurons as used in some cortical brain stimulation.

Another object and advantage of our invention is the possibility of better control the volume where the vagal nerve is, to stimulate the vagal nerve and only the vagal nerve, to control blood pressure.

If one or more of the cited objectives is not achieved in a particular case, any one of the remaining objectives should be considered enough for the patent disclosure to stand, as these objectives and advantages are independent of each other.

Further objects and advantages of my invention will become apparent from a consideration of the drawings, the summary, the description of the invention and its variations, and the claims.

### SUMMARY

It is well known in cardiology that the heart pumping efficiency is a direct consequence of a proper propagation, in time and space, through all available electrical paths in the heart cells, of the electrical pulse that causes the heart contraction, including the contraction sequence. Included in these cells are the cells of the miocardium, the cells of the His bundle, of the right and left bundle, of the Purkinjie fibers and others. This is acknowledged to be true whether the electrical pulse is the natural one starting at the SAN (sino-atrial node) or an artificial one, starting at the anchoring position of an artificial heart pacemaker—whether it is a single electrode or multiple electrodes. It is interesting to note here that evolution does not, and in fact cannot progress along modifications on the heart design toward the most efficient possible pumping, but only to the most efficient pumping starting from the existing configuration—which may well be incompatible with modifications the best solution. It is not true at all that the heart that has been evolved by natural selection to the best solution—and in the case of the heart contraction to the most efficient pumping contracting sequence. Moreover, even if nature had evolved the best possible contraction sequence, the artificial heart pacemaker does not inject the electric current at the same location as the natural pacemakers, and consequently a well designed artificial heart pacemaker needs to correct for this variation—while the current devices do not correct. Finally, due to the asymmetry of the heart muscle, it would not be expectable that the currently used symmetric electrode would best substitute the natural pacemaker. Consequently, what is needed is a heart pacemaker that could maximize the pumping efficiency. Such a goal has eluded the practitioners because of a lack of mechanism for precise control of the path of the injected electric charge, in position, direction and relative timing. Our invention is a step in the direction of better control of the path and timing of this stimulating pulse. Our invention discloses a mechanism to control the magnitude and the direction of the initial current injection in the heart muscle, also time delays between current injected from different locations on the surface of the stimulator even after the current is injected in the heart or other organs; in other words, our invention affords the possibility of controlling what we call the “vector current”, and the relative time at different directions and places, as opposed to only its magnitude, as in prior art. Our invention also applies to other electrical stimulations as brain (DBS and cortical stimulation), neurons, spine, skin, cochlea and others.

### DRAWINGS

FIG. 1. Major parts of the normal human heart.

FIG. 2. Two electrodes for Cardiac Resynchronization Therapy with multiple supercapacitors at several positions along the cables.

FIG. 3. A heart-type electrical stimulator (artificial heart pacemaker or piquita). 140-t1 points to type1 or active electrodes and 140-t2 points to type2 or passive electrodes.

FIG. 4. Shows a perspective view of a picafina brain-type stimulator of our invention showing a schematic view of the inner wires and connections. Not all wires in the vertical direction are shown, for simplicity, but only the wires that connect to the top layer of electrodes plus a few more to lower layers. This embodiment uses one dedicated wire for each electrode and both type-1 140_{—}t1 and type-2 140_{—}t2 electrodes.

FIGS. 5(a, b and c). Three examples of electric field lines (which are the lines along which a positive charge would move). The field lines differ for different electric charges due to their sign (+ or −), numerical value (q, q/2, etc.) and position in space. The reader should notice that such slightly differences in charges produce vastly different shapes of the electric fields, which are the paths of charges free to move in the space in each configuration. The space may be around a heart, for example, so each charge distribution causes a different heart contraction sequence, because the electric charges would move following a different path (causing different muscles to contract) and at different speeds, causing a time delay characteristic of each speed—as much as different cars moving towards different directions and at different speeds would arrive at different places and at different times.

FIGS. 5 (d and e). Effect of changing the numerical value of the electric charges, which is equivalent to modifying the electric potential (or voltage), with the same spatial configuration of two positive and one negative charges at the same locations. The reader will notice how vastly different the field lines are with a simple change of one charge from a small value of q/10 to a larger value of 2.5 q.

FIG. 6. A heart-type electric pacemaker (piquita) with multiple shaped electrodes connected to the associated battery and electronics. The larger number of connecting wires 124 is for the embodiment with a dedicated wire to each electrode. Other embodiments may have multiple electrodes connected to the same wire together with a selecting mechanism to select the electrodes that are connected to the battery/controlling electronics.

FIG. 7. shows a variation of the heart stimulator piquita. In this embodiment electrodes are only present at the tines 131. Some of them may be of type-1, some others may be of type-2, a difference that is not made in this particular figure.

FIGS. 8a, 8b and 8c. Resistor network similar to current path in cells but with denumerable paths.

FIGS. 9a, 9b, 9c and 9d. Simplified, cartoon-like representations of the right part of a human heart showing four sequential stages of squeezing the atrium. The blood is pumped down. The blood level at the ventricle 310_ventr is indicated by the raising bl. The atrium 310_atr keeps contracting from top to bottom, therefore squeezing the blood down through the one-way tricuspid valve 307. The left part is essentially the same.

FIGS. 10a, 10b, 10c and 10d. Simplified, cartoon-like schematic representations of the right part of a human heart, showing four sequential stages of squeezing the ventricle 310_ventr. The blood level bl is now fixed at the top of the ventricle 320_ventr, which keeps contracting upwards from the bottom, forcing the blood out of it. The squeezing of the ventricle 310_ventr is grossly exaggerated, as a normal heart squeezes only 55% to 70% of its blood volume out, and the squeezing is not as neatly sequential as indicated in the figure, which exaggerates the situation for better observation. Yet it is worth to mention that such an exaggerated contraction sequence as shown would be closer to a better peristaltic pump than the heart we were given.

FIG. 11. Shows a schematic representation of a brain-type picafina of our invention, with some of the electronics that controls its functioning. Similar designs apply to the heart-type piquita and other variations. This figure shows both type-1 and type-2 electrodes.

FIG. 12. The gravitational field of the planet Earth showing an exaggerated sideways deformation due to mountain m.

FIG. 13. Shows a version of our invention designed for Deep Brain Stimulation (DBS), which is often used to control Parkinson's Disease and essential tremor.

FIG. 14. Shows a schematic connection between the sealed box 110 containing the energy storage unit BAT1 (battery, etc.), the microprocessor MP1, the necessary electronics, the electrical connecting means and a brain-type picafina stimulator of our invention. Similar connections are valid for the heart-type piquita and other variations.

FIG. 15. Shows a schematic representation of a brain-type picafina of our invention, with some of the electronics that controls its functioning. Similar designs apply to the heart-type piquita and other variations.

FIGS. 16a and 16b. Shows two variations of electrode shapes: octogonal and hexagonal, with filling electrodes of different shape as needed.

### DETAILED DESCRIPTION

In the following we are introducing some terms with precisely defined meaning which we clarify here: these are passive electrodes, active electrodes, supercapacitors, picafina, piquita, planarium. Passive electrodes are electrodes which are capable of creating electric field lines, but not capable to inject electric current in the space surrounding them. Physically, and this is most important for this patent, what characterizes an electrode as passive is that it is covered by an electric insulating layer. The insulating layer prevents any electric charge from leaving the electrode to the outside of the supporting structure, whether a picafina, a piquita, a planarium or any other type. The reader should keep in mind that electrical insulators do not prevent the electric field lines from existing past the insulating layer covering the passive electrodes, but only prevent the electric charges from penetrating them, and, consequently, the passive electrodes are perfectly capable of creating field lines in their surrounding volume, e.g., in the heart muscle. Aside from this, passive electrodes work best for their purposes the larger the electric charges they can hold, because the electric field is determined by the value, or magnitude of the electric charge. Consequently the passive electrodes should be capable to store the maximum amount of electric charge. They are preferentially larger capacitors, generally of the class known as supercapacitors, because what is meant by “capacitance” is the capacity to hold electric charge. In this account the passive electrodes are largely different than the electrodes in use today, because the passive electrodes are made to maximize the charge stored, as it will become clear as their function is further discussed. They are labeled as 140_{—}t2.

Active electrodes are the ordinary electrodes of the electrical stimulators used by current devices: they are capable of injecting electric charges in the space surrounding them, and these charges are then capable of moving in the medium surrounding the electrodes. They are labeled here as 140_{—}t1.

Picafina is a elongated penetrating supporting structure of cylindrical shape, of the type generally used for Deep Brain Stimulation, measuring a few centimeters in length by one-plus millimeters in diameter (for example, 7 cm in length by 1.3 mm in diameter), which encompass other forms of stimulators too. Picafina is what is usually called by the companies by the general term “lead”, which we avoid for being misleading. The picafina of our invention typically has several active stimulating electrodes at its extremity and passive electrodes distributed over its extremity and over its whole body as well. Cf. with piquita and planarium.

Piquita is a penetrating supporting structure which often resembles an anchor (as a shipping anchor), with a small penetrating structure with some few anchoring tips, called “tines”, which serve to prevent the piquita from dislodging from the tissue where it has been inserted. Other shapes are possible too, and we use the term to indicate electric stimulators designed for use in the heart. Piquita is designed to be used for stimulation of the heart, where it is implanted on its inner side where it is inserted from a vein, typically the sub-clavian vein, with no harm to the person who receives it. It is currently also referred to as a “lead”, in spite of its form being so different than the brain implant. Cf. with picafina and planarium.

Planarium is a structure which is designed to support a number of stimulating electrodes on a planar supporting structure which may be attached to the skin of a person or to the outer surface of an organ (as to the pericardium of a heart or the outer surface of a brain). Cf. with picafina and piquita.

Supercapacitor is an ill-defined term used in the electronics world to indicate an ordinary capacitor typically made with late 20^{th}-Century technology, typically, but not exclusively involving a large number of small cavities in the bulk of the material, with the consequence of a very large increase of the surface area of the material, and typically meaning a capacitor with capacitance measured in Farads, which was beyond the wildest dream a few decades ago. We cannot define the term better than the electronics usage of it, but for us the term means what a typical electronic engineer would consider a capacitor with a truly large capability of storing electric charge, typically, but not restrictively so, in the value of several Farads. The larger the electric charge capability the best. When necessary, they are labeled as SC1_1, SC2_1, etc., but often they are just referred by their functions as passive electrodes and then referred as 140_{—}t2. Preferably passive electrodes are more than a metallic surface covered by an insulator; under the insulating layer it is preferable to have a supercapacitor than just a metallic surface.

Tines are anchoring arms, generally at the tip of the piquita (heart electric stimulator), similar to the grabbing arms at a ship's anchor, which serve the same purpose as the ship's anchor: to prevent the piquita from dislodging from its insertion place in the heart. Many piquita in use today have four tines but this number is not required.

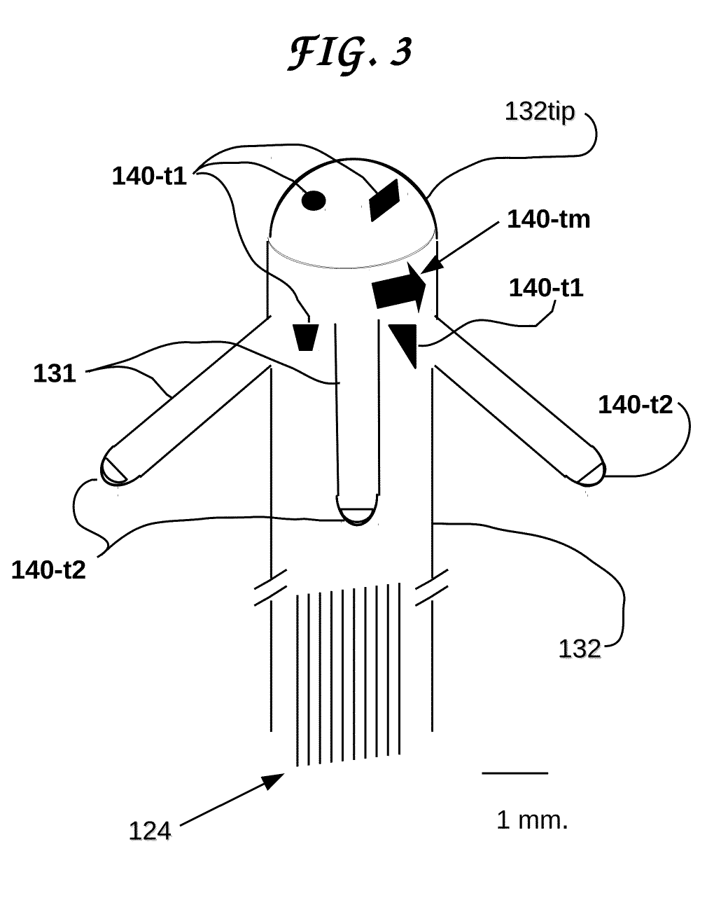

FIG. 3 shows the main embodiment of our invention, which is for heart pacemaking applications, which we call piquita. FIG. 3 shows one of the current art anchoring distal extremities 132tip of a current art heart pacemaker with the improvements of our invention. Note that different ending anchoring attachments 131 are in use, and that the model shown in FIG. 3 uses one of the several used attachment endings, but the same principles apply to other anchoring attachments. The anchoring devices 131 are known as tines. The main body 132 of the piquita device may have a diameter of 3 mm or less, as 2 mm or 1 mm (approximate dimensions), and the smaller anchoring side arms 131 may have a diameter of 1 mm or 0.5 mm (approximate dimensions, the actual dimension being unimportant to the invention). Anchoring arms 131 should have such size and strength enough to keep the tip of the stimulating piquita structure 132 secured in place once it is inserted into the heart muscle from the inside of the heart. Anchoring arms 131 should prevent the piquita stimulating device from moving back, out or the muscle, this being one of the reasons for its shape and form, resembling a ship's anchor, which has the similar function of holding firm to the sand below the ship, or the arrow's tip, which holds the arrow inside the hapless person or animal onto which it has been thrown. These dimensions may vary without changing the nature of our invention and these values are given as a possible dimensions only. On the surface of the main body 132 and of the smaller side arms 131 there are several random-shaped patches which are represented by either a solid black or a white shape represented by its contour. The solid black odd-shaped patches 140-t1 represent electrodes which we call active, or type-1 electrodes, and the open, odd-shaped patches 140-t2 represent electrodes which we call passive, or type-II or type-2 electrodes. These type-2 electrodes is one of the main inventive characteristic or our invention. They are also described in our patent application Ser. No. 13/470,275, currently allowed, which discloses a more complex embodiment of the invention disclosed here. The invention disclosed here does not use the local addresses near the electrodes, having instead a large number of wires connecting the electrodes to the battery BAT1/controlling electronics-microprocessor MP1, one dedicated wire for each electrode. The invention disclosed here has one less element than the invention disclosed in Ser. No. 13/470,275.

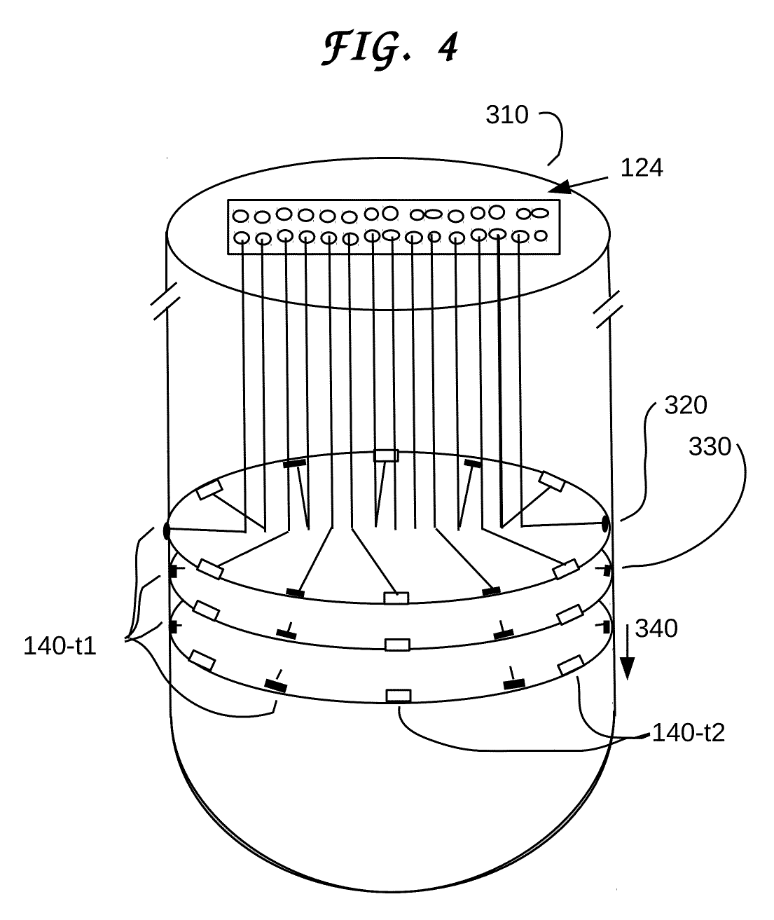

FIG. 4 shows a perspective view of the brain-style (a.k.a. Picafina), with some wires down the length of the device, but not all wires to prevent cluttering the drawing. Only the wires that make the connection to the electrodes at the top layer 320 are shown. Other electrodes, on the layers below (330, 340, etc.), are also connected to dedicated wires, similar to the ones shown in this figure. In the main embodiment the wires are of the printed circuit type, but lose wires are also possible, though a smaller number of them would be possible. At the top of the brain-type picafina shown in FIG. 4 there is an electrical connector, which is capable of matching another connector (as male-female type) with wires leading to the battery BAT1 and controlling electronics/microprocessor MP1 implanted at another location, inside sealed box 110, as in devices in use today.

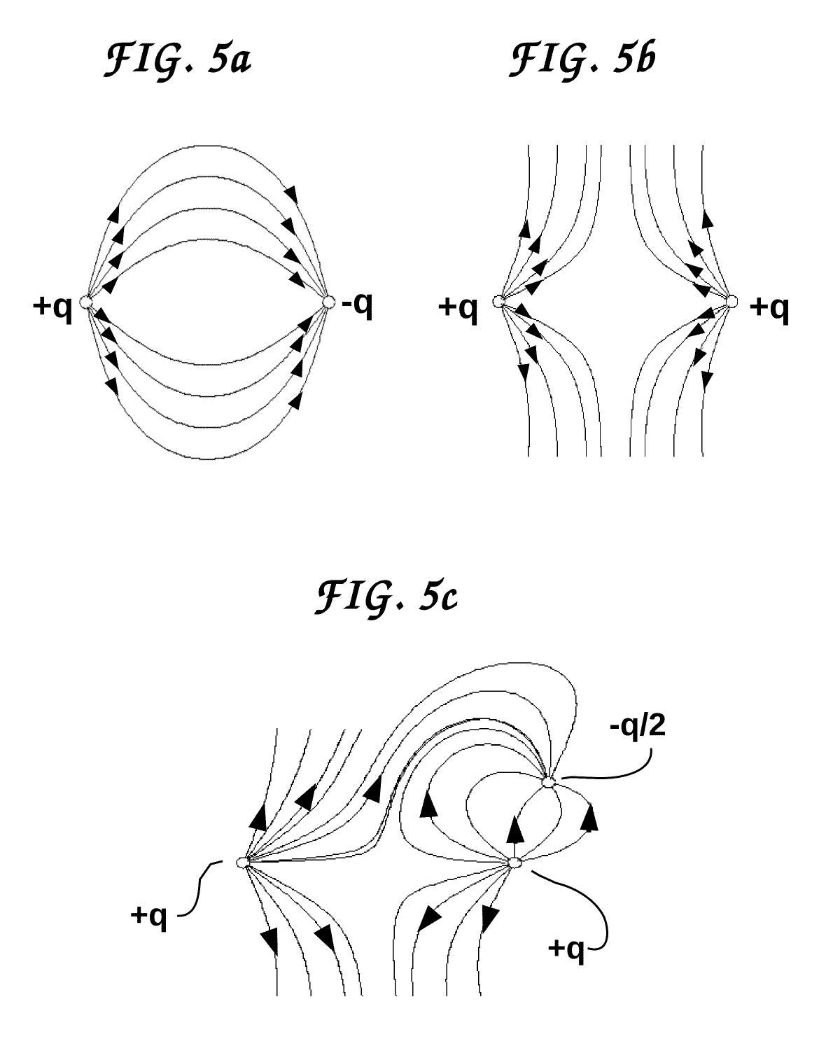

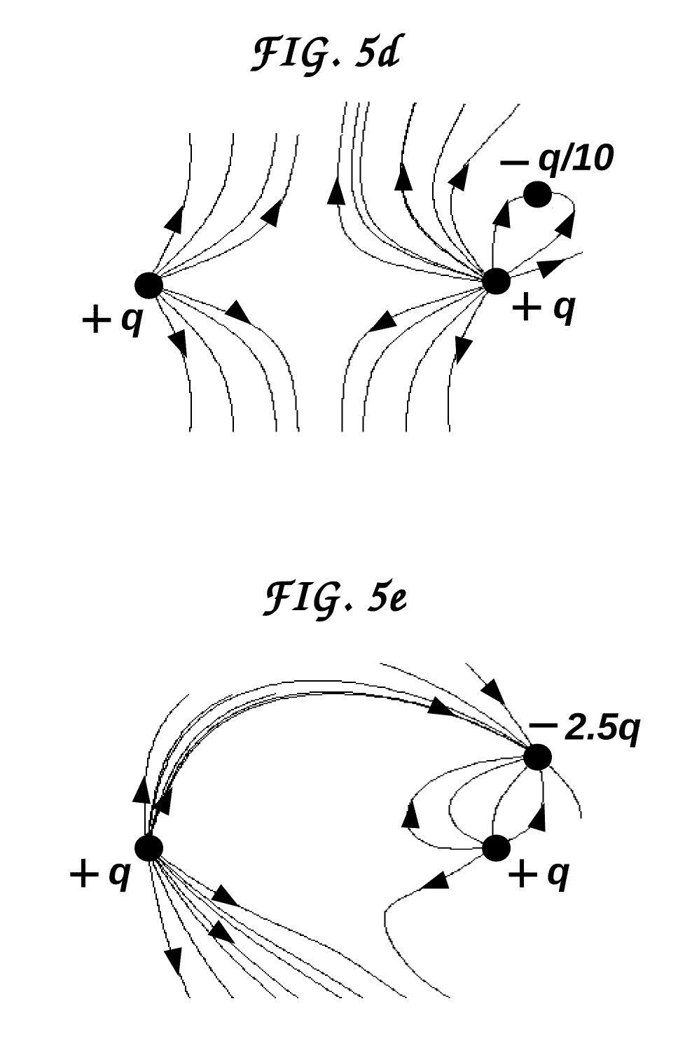

Active, or type-1 electrodes 140-t1 have a metallic surface which is capable of conducting electricity. Other than their smaller sizes and odd-shapes, they correspond to the electrodes in use today (prior-art electrodes in patent jargon) for electrical stimulation of the heart, brain, and other body parts. It is worth to mention that though the size and configuration of the electrodes disclosed here add to their functionality, part of the improvement disclosed here is also achievable with larger electrodes as used by current electrical stimulators. Passive, or type-2 electrodes 140-t2 besides being preferably made with supercapacitors, their surface is covered by an insulating layer, which, in the main embodiment is made of silicon oxide. It is worth to mention that passive electrodes may be simple metallic surfaces covered by an insulating layer, totally similar to active electrodes, just that they do not function as well. Passive, type-2 electrodes are unable to inject current into the surrounding tissues, but when set at fixed electric potentials (voltages) they do change the shape of the electric field in the neighborhood of the piquita, therefore changing the paths of the injected currents. Passive (type-2) electrodes are incorporated in the piquita for the purpose of field shaping (to change the spatial configuration of the surrounding electric field which in turn changes the path of the electrical stimulation). Examples of field-shaping are shown in FIGS. 5 (a, b, c, d and e), which display several different electric field configurations for different electric charge distributions.

As stated above, the electric field is a function of the electric charges at different places, not of the voltages at the places or a function of the current emanating from the places, so the passive electrodes are preferably, but not necessarily constructed with the technology of supercapacitors (see definition above) to maximize their impact on the electric field created by them. This is so because the electric field is governed by Coulomb's law:

E(vector)=k*(Q/r̂2)(r-hat), (EQ_Efield)

Where E is a vector (emphasized by the mathematically unconventional word in parenthesis following it), k is a constant of proportionality described in most elementary books on electricity and magnetism, Q is the electric charge which creates the electric field, r is the distance (scalar, just the number) from the charge Q to the point where the field is calculated, and r-hat is a unit vector, which gives the direction to the field E on the left-hand-side, without affecting its magnitude, known as unit vector. In mathematical texts r-hat is indicated by a bold-face r with a hat on top of it to indicate it is a vector of unit length. There are conventionally accepted norms governing the direction of E and other peculiarities of the vector E which we are swiping under the rug for conciseveness.

Observing the Coulomb's law above it is seen that the electric field E is directly proportional to the electric charge Q, so the larger the charge Q the larger the electric field E is. Since the force F on the charge injected by the active electrodes is proportional to the electric field E, it follows that a larger electric field E causes a larger force F on the injected particle and can, therefore, have larger influence on its motion (as a larger engine car offers more options to the driver when compared with a smaller engine car).

Now, the value of the charge that a particular battery can “pack” into the electrode depends on the “force” or “strength” of the battery, which is measured by its electric potential (unfortunately called voltage in US) and a few geometric and space characteristics which are lumped in a quantity called capacitance, usually indicated by the letter C (capital C):

Q=C*V

It follows that one can arbitrarily increase the charge Q that creates the electric field E (and consequently the force F on the electric charge injected by the active electrode) by arbitrarily increasing V or increasing C. Unfortunately V is created by a battery, so it cannot exceed the electric potential of the battery, which normally is a few volts only, as known by most of us (just think that most of the batteries we ever handle are 1.2 V or 1.5 V, the 12V car battery being actually six 2V individual batteries (called cells in this case) on “top” of each other to add to 12V). Consequently, given that V in the equation above is limited to a rather low value, it is left to see if C can be increased. It turns out that until recently C was also rather restricted in maximum value, but recently a new class of capacitors, baptized as “supercapacitors” do sport an enormously large value of C. In the case of the supercapacitors their capacitance value (C) is increased using modern technology that largely increases the surface area of the device, a porous surface under the insulating layer.

The part of the main embodiment of our invention which preferentially (but not exclusively) uses a supercapacitor for passive electrodes does so to boost the numerical value of the stored charge “Q”, which in turn increases the value of the magnitude of the electric field E, which in turn increases the magnitude of the force F imparted on the electric charge injected by the active electrode. Of course that not necessarily a very large value of F is required, particularly all over the place. What our invention offers is the possibility of a large force F when one such is needed. Moreover, for the same required force F a much smaller electric potential (voltage) V is required if the passive electrode is characterized by a large capacitance C, therefore decreasing the requirements on the valuable battery that nobody wants to replace (with a surgery!).

Another part of the main embodiment, with the same objective of increasing control on the electric field E is the introduction of the type_{—}2 passive electrodes 140_{—}t2 with a large plane shape which we call planarium. These electrodes, possibly using the supercapacitor technology, but not necessarily so, are manufactured in the necessary shape to be implanted in different parts of the patient's body. The planaria differ in shape from the picafina and piquita in that planaria are generally flat, perhaps with a curvature to conform to a shape but still sheet-like in general shape. For example, a planarium could be manufactured to cover the outer contour of the heart, just outside the pericardium, which is the sac that contains the heart. We call it the pericardium planarium. Such a planarium would have a strong control on the electric field inside its volume, but it would require an open-heart surgery to implant, which is highly undesirable. It may become more acceptable in cases where, for some reason, the patient is already undergoing open heart surgery anyway, in which case the pericardium planarium would be desirable. Other designs are less invasive than the pericardium planarium, but also less able to control the electric field in the heart muscle.

Another option is to implant planaria just below the skin, at the chest, side of thorax and back of the patient. We call these under skin planaria. These would require less invasive surgery, at the cost of being less effective for being more distant from the heart than the pericardium planarium. One or two under skin planaria could be implanted near the implantation of the sealed box 110 at the initial implant time, using the opportunity that the patient is already opened up anyway.

Another option, which is still less effective than the under skin planaria is to make passive electrodes attached to a tight shirt-like cover for the thorax, as a tight T-shirt, which is connected to a battery conveniently located, say, at the bottom of it, connected by wires to the electrodes at the inner surface of the shirt-like wearable device. We call this the shirt-like planarium. The shirt-like planarium offers still less effective control of the electric field, but they offer the advantage of requiring no surgery at all, including for battery changes. This shirt-like planaria could be just at the front of the chest, or just at the sides of the torax, or just at the back, or any combination of these.

Still another variation of shape for the type_{—}2 electrodes 140_{—}t2 is to manufacture type_{—}2 electrodes on the length of a wire which is designed to be inserted into the chest using laparoscopy (that is, minimally invasive surgery through a small hole in the body). The effectiveness of such a line of type_{—}2 electrodes would be even less than a under-skin planarium because it would offer less surface area, but it would offer the advantage of closeness to the heart. If effective surgical techniques were developed to perform such a surgery it may become a good choice due to laparoscopy being minimally invasive surgery. In principle techniques could be also devised to insert a planarium via laparoscopy too, probably not to cover the whole heart as the pericardium-type planarium, but still stretching near the heart on some of its sides or in front or back of it.

As the reader will see, several types of planaria-type electrodes can be devised to control the electric field in the heart muscle with different effectiveness and different levels of difficulty and surgical danger that have to be weighted by the surgeon and by the patient on a case-by-case basis.

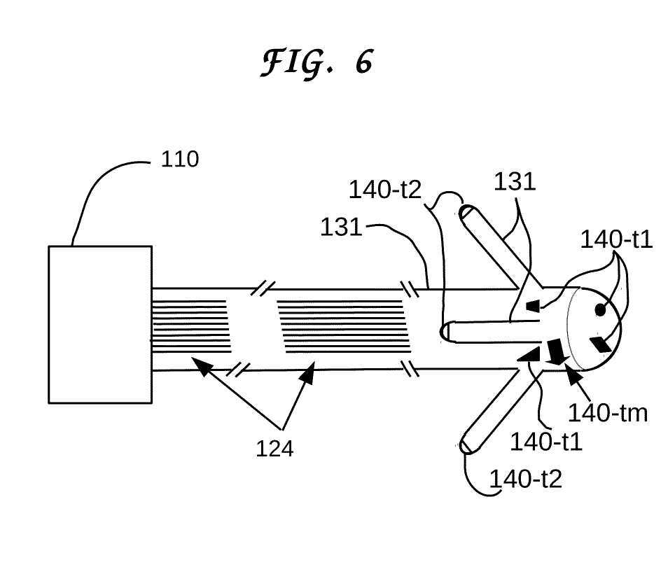

The invention also discloses an important marker to determine the angular position of the piquita with respect to the heart (or brain, or nerve, etc.) in which it is implanted. FIG. 3 and FIG. 6 shows one such possible marker: a type-1 active electrode 140-tm with such an X-ray opacity (absorption or scattering cross-section) to be visible during the fluoroscopic images taken during electrode implantation as normally done. Other markers are possible for the same purpose, as the same shapes on type-2 passive electrodes, as side arms 131 of different lengths and/or diameters, or any other asymmetric feature that is visible in some sort of imaging technique, as MRI, X-ray, ultrasound, etc. It is part of our invention that each electrode position and size and orientation is known to the cardiologist (and the computer which he will use to program the device), each electrode being know by a number, as 1, 2, 3, . . . etc., or any other identifying pattern. Marker 140-tm allows for the computer program to know the angular position of each electrode, which is needed to determine which individual electrode to connect to which voltage, according to their actual position within the heart muscle, as the piquita happened to have been anchored in it.

Inside the main body 132 and the side arms 131 of the piquita supporting structure, there are wires 124 extending from the controlling electronics, microprocessor and battery to each electrode 140 (of either type, t1 or t2). Wires 124 may be either standard wires or may also be printed wires, as in printed circuit boards, in this case more likely printed on a flexible plastic support but any of the existing technologies are acceptable, this patent being not on the printed circuit technology. The technology of printed circuits is a well advanced technology with many methods to print the wires, and the wire manufacturing is not part of this invention, as any of the existing technologies are acceptable to implement the invention.

The main embodiment uses 10 wires from the battery pack/control unit housed in sealed box 110 to the piquita supporting unit 132, which are connected to the 10 available electrodes 140 by the 10 wires 124—one wire for each electrode 140. This particular choice of 10 wires and 10 electrodes should not be taken as a limitation on the invention, because more wires and electrodes, or less wires and electrodes are possible, still within the scope of the invention, as obvious to people familiar with electronics. It is also possible to connect the ground (or return) wire to any number of electrodes (or pads), both type-1 and type-2.

The random placement, shape and size of the electrodes is a distinct feature of our invention, as it contributes for the creation of a spatial asymmetry of the electrodes, which in turn causes an asymmetry in the spatial distribution of the injected current, either its magnitude or its direction or both. Careful selection of which electrodes to turn on, and at which electric potentials (voltages), can create the most desirable electric field shape on the volume of the heart. As the reader will remember from the above explanations, the electric potential chosen by the medical practitioner or by the patient determines the charge on each passive electrode, depending on their particular value of capacitance C, and this charge, in turn, determines the value of the electric field E, which in turn determines the motion (time of arrival and path) of the electric charges injected in the heart. A careful selection of which electrodes to turn on, is able to produce a better resulting stimulation which is suited to the asymmetric heart muscle 3-dimensional shape and causes a more complete squeezing sequence and better ejection fraction (the fraction of blood sent out of the heart). It is to be noted that if any symmetry is required, our invention is backwards compatible, being able to reproduce old art stimulating surfaces as a particular case of an arbitrary shaped surface. Naturally the degree of symmetry possible to be achieved depends on the number of electrodes available: more asymmetry with more electrodes (that is, more complex electric fields with more electrodes).

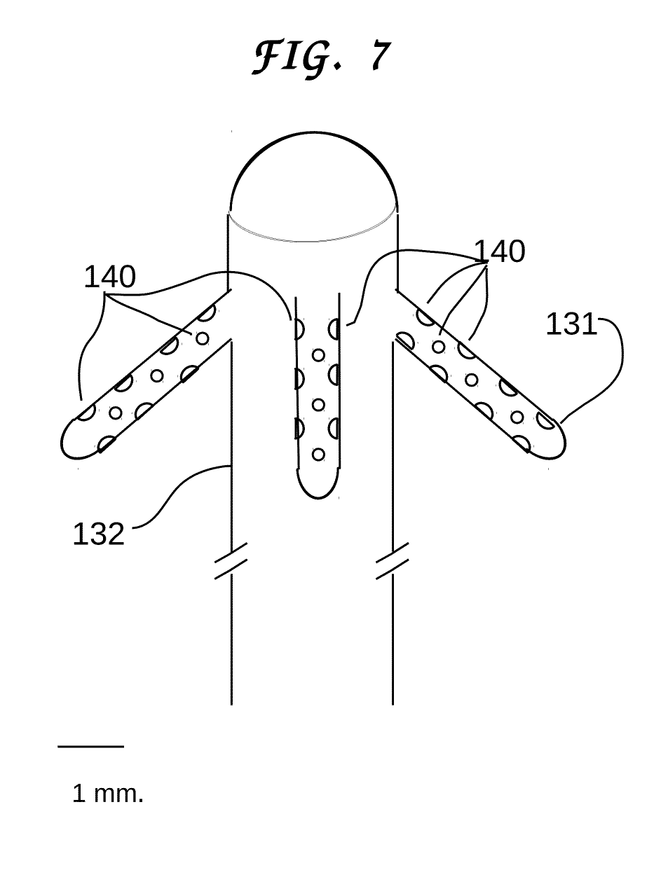

FIG. 7 shows a variation of the heart-type stimulator piquita with electrodes only at the surface of the side or anchoring arms 131.

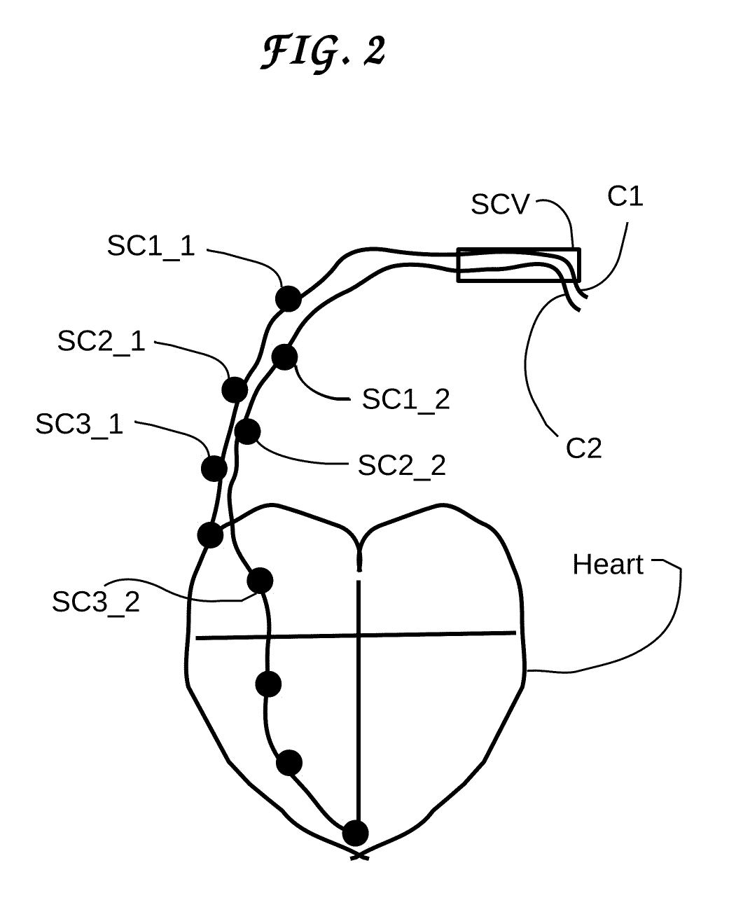

FIG. 2 shows one of the features of the main embodiment of this invention, which is the supercapacitors SC1_1, SC2_1, SC3_1, etc along the wires or cables C1 and C2. FIG. 2 shows a heart with its 4 chambers and two wires or cables C1 and C2, on which a number of passive electrodes 140_{—}t1 are located. In this case the passive electrodes 140_{—}t2 are made as supercapacitors SC, but variations with simple metallic surfaces for 140_{—}t2 are possible. Supercapacitors SC, are one of the possible incarnations of the passive electrodes 140_{—}t2. Generally speaking, the passive electrodes are distributed over as wide a volume as possible, within the constraints of the surgery and the location of the device, to have more control on the electric field, described by equation (EQ_Efield), as will be understood by the electrical engineers and physicists. In the case indicated in FIG. 2 they are distributed over the length of the wire/cable C1 and C2. The wider is the distribution of the passive electrodes 140_{—}t2 over the body of the patient, the stronger is the control that the microcontroller MC1 has on the value and direction of the electric field that eventually control the direction and speed of the electric charges injected in the heart (or other body part). The passive electrodes 140_{—}t2 (which in the main embodiment are supercapacitors SC) preferably should be located near the volume where the electric charges are moving. This is a consequence of the mathematical dependence of the electric field E (see equation (EQ_Efield)) on the distance to the place where the field is, which decreases with the square of the distance, so, for passive electrodes located at large distances from the desired location the contribution is smaller than the contribution by another passive electrodes located closer to the desired location.

### Operation of the Invention

Background Information on Operation of the Invention.

Knowledge from two distinct fields are necessary to understand our invention. Firstly it is necessary to understand the mechanism of heart pumping from the cell/muscle point of view, usually an area studied by medical people, cardiologists and electrophysiologists. Secondly, it is necessary to understand the mechanism of propagation of the electrical charge that is associated with the contraction of the cells that make the heart muscle, usually an area of knowledge studied by physicists and engineers. Since this invention involves knowledge from two so different fields of knowledge, namely electrical engineering & physics and medicine & physiology, each part of the description needs to be detailed enough to be understood by someone with little or no knowledge on that part, whether it is an electrical engineering concept, unfamiliar to a medical person, or a cellular physiology concept, unfamiliar to an electrical engineer. In any case, the inventors prefer to strictly follow the intent of the patent disclosure, which is to be thorough and complete on the description of the device to make it easier for others to do it all again.

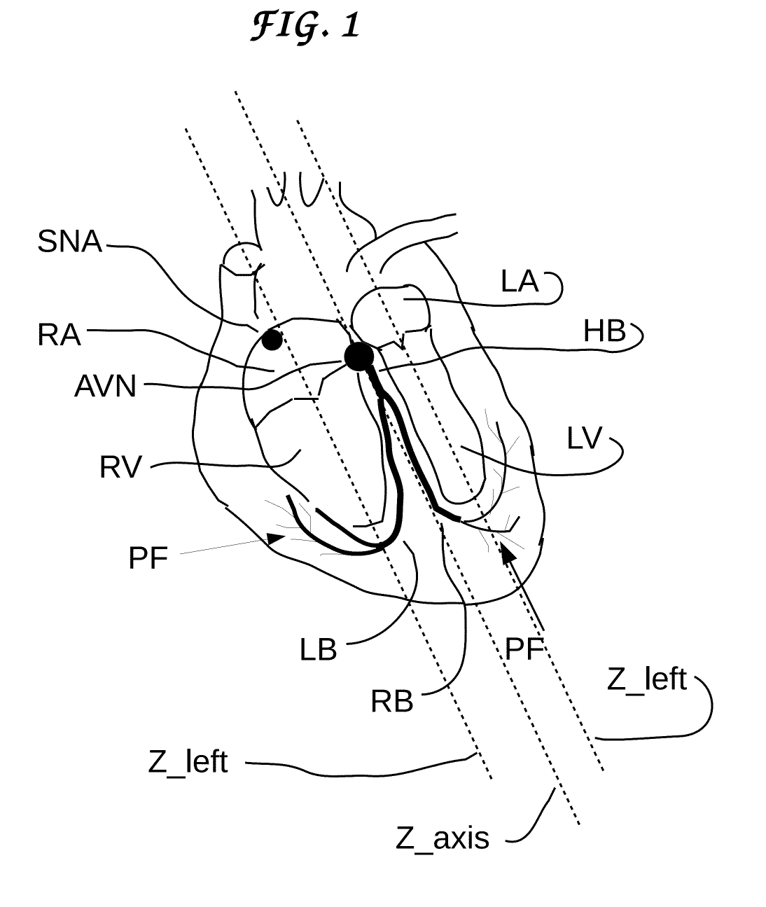

FIG. 1 shows the major parts of a human heart. The heart is divided into four chambers: left and right atria, at the upper part of the heart, and left and right ventricles, at the lower part of the heart. Right and left are arbitrarily assigned to be from the point of view of the person where the heart is—which is the opposite left-right from the point of view of the observer looking at the person from the front. The atria are more holding chambers then actually pumping devices, evolved to quickly fill up the ventricles, below them, and consequently their walls are thinner when compared with the lower part, the ventricles. The right heart is responsible for the pulmonary circulation, receiving venous (non- or little-oxygenated) blood from the full body at the right atrium RA, passing it down to the right ventricle below it, from where the blood is pumped to the lungs. This corresponds to a short path, to the lungs and back. Back from the lungs, the blood enters the left atrium LA, which holds some oxygenated blood, then releases it down to the left ventricle LV below it, from where the blood is then pumped to the whole body. The left heart pumps blood to the whole body, which involves more work when compared with the shorter path from the right heart to lungs and back, so the left ventricle has thicker, stronger walls. These facts related to the heart wall thickness are known to all medical practitioners, but its consequences are largely overlooked, particularly its implications on the electric pulse propagation in the heart muscle and its consequence, the contraction sequence, so the reader is encouraged to think on it: to think of the implications of the differences of the local electrical resistivity of the heart muscle at different parts of it, even at birth, differences that ought to accentuate as the heart ages and its muscles change as much as the muscle (of fat) in the belly changes or the skin under the eyes change. These considerations on the wall thickness and composition are of importance for our invention, because our invention deals with the optimization of the pumping mechanism of the heart, which is heavily dependent on the propagation delays and on the trajectory of the electrical pulses that causes the heart cell contraction and consequently the pumping mechanism, as explained below. The pumping optimization has also to do with the local resistivity of the heart muscles through which the electric pulse propagates, which changes with time throughout the lifetime of the person. The local resistivity is most important for the correct muscle contracting sequence, as it will become more clear further down when we discuss the electric pulse propagation that is responsible for the contracting sequence.

The heart being a little taller (that is, along the person's vertical direction) than wider, let us further define a “vertical” axis on the heart as an axis passing through its almost vertical direction, even though the heart is neither quite vertically aligned nor that much “taller” than “wider”. We will call this the heart z axis Z_axis at its center, with two other axis through the centerline of the left and right sections of the heart, which we call Z_left and Z_right.

As is known by the medical people, and particularly the electrophysiologists, the heart contracts sequentially in the sense that each chamber initiates a contraction sequence at the extremity that is opposite to the exit port, then progressively contracting cells that are located closer to the exit port, until the cells most near the exit port, when the cycle completes and stops. One such cycle occurs at the upper chamber (the atrium) followed by another such cycle at the lower chamber (the ventricle), then the whole process repeats: atrium-ventricle-atrium-ventricle . . . . For this cycle to occur in an optimal sequence, the electric charges that propagate through the heart muscle must propagate at equal speeds all around the heart, so that they advance together all around the heart, front, back, left and right, around the z_left (or z_right). Moreover, each part of the heart hopefully contracts with a strength proportional to the required force for the particular chamber and the level of blood supply needed at the moment, which depends on many physiological factors, as physical activity, emotional stress, and so on. For the ideal heart the current density should proportional to the local required pumping strength—but it is not so in the real heart. Again for an ideal heart, the wall thickness should be proportional to the required pumping strength—this is approximately true, the ventricules being thicker than the atria, and the left ventricule having thicker walls than the right ventricule. Finally, the ideal heart, should have a contraction equally strong all around the heart, that is, at the same values of the coordinate along z (left and right, the z_left/z_right axis), so that there ought to exist a higher electric current where the heart wall is thicker, because there are more cells to stimulate there, causing the same current density (the same force). Unfortunately most hearts, even very good ones, fail to keep a good electrical pulse progression, resulting in non-optimal heart contraction and consequently in smaller pumping fraction (smaller volumetric fraction of the blood pumped forward). Advancing the conclusion, our invention addresses this problem of muscle strength of contraction all around the heart with the objective of improving the volumetric pumping efficiency.

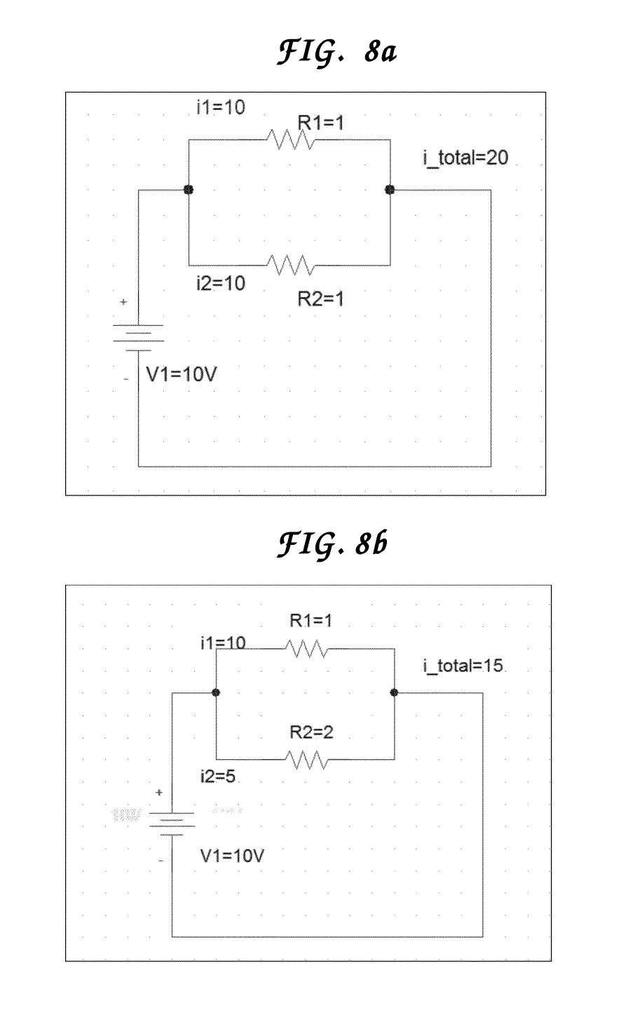

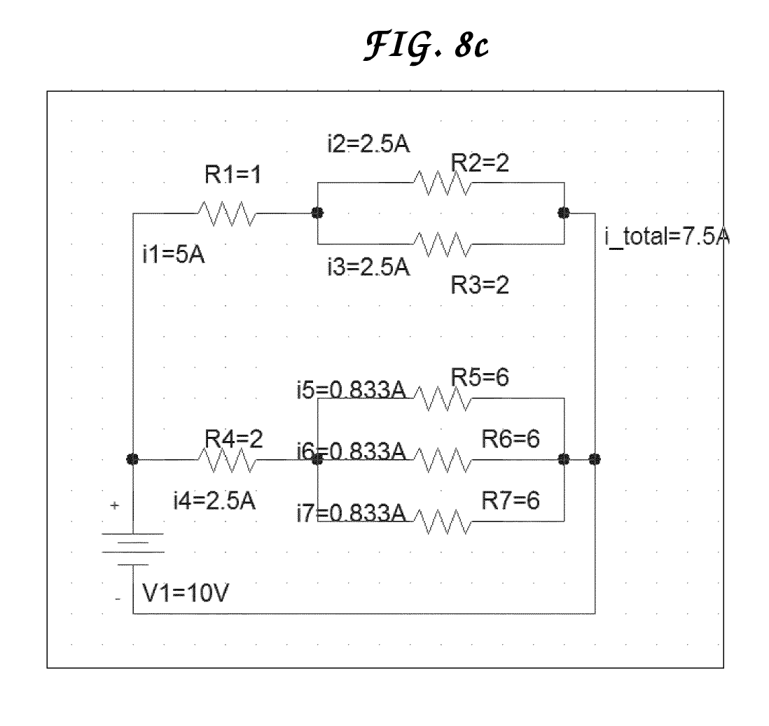

As a simpler example of the splitting of currents through different parts of the heart we invite the reader to go through a simpler current splitting, one that is usually part of the homework of standard high-school in Europe and South America. The numerical values were chosen to make the calculated values easier to manipulate, even if unrealistic, avoiding micros, millis, kilos and megas, for the benefit of the readers with less technical background. The technically minded reader is asked not to get disgusted by the unrealistic high values of currents and the unrealistic low values of resistances. FIGS. 8a, 8b and 8c show three resistors networks connected to a battery. FIG. 8a shows two resistors of equal value, R1 and R2, in parallel (1 Ohm each) connected to a battery with e=10V. Standard electrical network calculations shows that the current in each resistor is 10 A (we omit the calculation here for sake of space and focus). The resistors being equal in value, it is expected that the currents through each is the same, as calculated. This is the situation of currents distributing throughout a perfectly symmetric heart, not a realistic heart. FIG. 8b shows two resistors of different values, R1=1 Ohm and R2=2 Ohms. In this case electrical network calculations calculate that the current in R1 is i1=10 A and that the current in R2 is i2=5 A. Common sense cause that one expect that R2 being twice as “hard”, or twice as “difficult”, should allow a current that is half of the current in R1, as calculations predict. This is the situation of currents distributing throughout a little more realistic heart, the left side of which using more current than the right side of it. Then, finally FIG. 8c shows a little more complex network with currents as shown at table T1, where the subindex of currents match the subindexes of resistors, that is i_1 is the current through R_1, etc. This corresponds to a more realistic approximation in that there are current subdivision on top of current subdivisions—again warning that current values in the heart are much lower, resistance values are much higher and the battery being less than 10 V, the numbers being chosen only to illustrate a concept, not to be realistic. The reader is now asked to extrapolate from this to a continuum situation, in which a battery applies an electrical force (a voltage, so to say) at the top-right of the heart, the sino-atrial node (SAN), which then spreads through the heart muscle, downwards, through not two resistors, as in 8a and 8b, not through 7 resistors, as in 8c, but through a network of zillions of a continuum of resistors, each offering a different resistance, according to the particular state of health of each cell, including their past history, as scars due to small infarcts, fat due to couch-potatoing, genetic defects and so on, so that the current in each cell is different from the neighboring cells. A different current would flow through each path, faster or slower according to the easiness or difficulty of motion, faster here, slower there, all the while the cell contraction occurring at the arrival of the electric charge with a strength proportional to the current i. Most people think about the contracting heart as an extension of FIG. 8a, with equal resistances at all paths, so the currents spread equally and flow at the same speed with an even contractions forward, but in the real heart the resistances are not equal, so the currents spread unequally and flow at different speeds with an uneven contraction sequence. This is the unstated assumption held by everybody that the heart contracts evenly, even though any cardiologist will immediately acknowledge that the cells at the miocardium ought to have very different electrical characteristics, there included their resistivity and their capacity to contract, the force that each can impart to a contraction cycle.

To understand the operation of our invention, the reader must keep in mind what causes the heart to contract, and therefore to pump the blood, and the sequential nature of this contraction, which is a consequence of the progressive motion of the electric charges through the heart walls, mostly the miocardium. FIG. 1 displays a human heart with the main parts indicated in it. Left and right are designations from the point of view of the person in which the heart is, which is the opposite of the viewer, facing the person. The right and left sections are responsible for two independent closed cycle blood flow: the right side of the heart pumps blood to the lungs then back is the side responsible for the pulmonary circulation, while the left side of the heart pumps blood to the whole body.

The heart muscle contraction occurs as a consequence of and together with the propagating electric pulse that moves in 3-D (three dimensions). As the electric charges propagate through the heart muscle, reaching new cells, each cell suffers a contraction event as the electric charges reaches it, one cell after another, in sequence. The moving electric pulse can be seen as a 3-dimensional extension of a falling domino event, each falling domino piece causing the fall of the piece ahead of it, the whole sequence propagating as a wave. The wave is easy to visualize in a domino falling sequence, because it is 1-dimensional, along the line of domino pieces, so I urge the reader to image such a wave propagating in 3 dimensions through the heart muscle and causing the muscle to progressively contract as the electric pulse propagates. This progressive nature of the electric charge motion and of muscle contraction should be kept in mind. Another analogy, this time in 2 dimensions is a circular wave on the surface of still water, propagating outwards from a point where a disturbance occurred, as a stone dropped on the water. The heart mechanism can be seen as a wave too, but in 3 dimensions. Moreover, the heart contraction is quickly followed by a decontraction event, which is the equivalent of the fallen dominoes raising up after the falling wave passes, which does not occur with the dominoes, or one can think as a person raising the domino pieces again after the wave passes.

The operation of the device is also due to the most important fact that the passive electrodes, for not injecting any charges in the subject, may be left on all the time which is not the case for the electrodes in current use. This continuous action is, in turn, needed to shape the electric field after the electric charges are injected by the active electrodes. This adds flexibility to the device, because the electric field shaping should occur even after the electric charge is injected, which occurs for a very short time. It is worth to note that existing electrical stimulators (heart pacemakers and other stimulators too) also shape the electric field (any charge or voltage distribution does create some electric field around it), yet the field shaping due to the active electrodes is not done on purpose to achieve the best heart contracting sequence (or other purposes for other devices), because the active electrodes exist for the purpose of injecting electric charges, not to shape the electric field. Another way to look at this is that the use of two types of electrodes allows our device to separate the two functions: electric charge injection and electric field shaping.

Besides the directional electric current flow, which is started again at every heart beat at the sinoatrial node, the local reactance plays a role, as it determines a 3-D continuous network which determines the time delay and magnitude of the local electric pulse, which in turn determines the local timing and strength of the local squeezing. Incorrect time delays of the electric pulse are costly for the pumping efficiency, because they are the very cause of the muscle contraction, that is, of the pumping, and localized higher or lower resistivity are costly too, because they change the electric current intensity, which in turn decrease or increase the strength of the muscle contraction, that is, of the pumping pressure, either way decreasing the total pumping volume. Our invention, designed to adjust the magnitude and the direction of the electric field throughout the heart muscle, corrects for these errors that accumulate throughout the life of the person, as the heart ages and changes. For example, in locations which, due to the changes that occurred throughout the life or due to genetics, the resistivity is larger (which decreases the electric current and its speed), they can be countered with a locally larger magnitude electric field. The reader can appreciate that if the left side of the left ventricle contracts first (while the right side not), then the heart would simply move to the opposite direction (to the right), as a whole, with no or little internal contraction. If then, later, the right side of the left ventricle contracts (while the left side does not, having entered in the quiescent part of its cycle), then the whole heart would move in the opposite direction again (to the left now), as a whole, and again with no or little internal contraction. In fact it is known that such a motion is common, as shown by many videos of an in vivo beating heart.

Taken together, controlling the direction and the magnitude of the current, our invention is capable of controlling the position and the magnitude of the squeezing sequence.

This electric wave propagating through the heart muscle starts naturally from an initiating point (the sino-atrial node or SNA in FIG. 1), which is located at the top of the right atrium RA. As explained above, the control of the 3-D electric pulse propagation through the heart muscle is the objective for the operation of our invention, as it will be seen in the sequel. This propagating electric pulse is known by the medical people as a depolarization wave, and the medical people associate a depolarization event to a muscle contraction event. This sequential contraction, characteristic of all peristaltic pumps, is similar to the process of squeezing toothpaste out of the tube: it is a progressive squeezing sequence which progress from the back to the exit port, as opposed to a simultaneous contraction from all sides as a person squeezing a tennis ball with one hand holding the ball for exercise. Granted that there are people that extract the toothpaste squeezing the tube from the middle, but it is universally acknowledged to be inefficient to do so, even by the very people that do it; they make a huge mess and drive other family members crazy trying to fix it all the time. The inventors suspect that many a marriage ended in divorce because of such improperly squeezed toothpaste tubes. It would be ideal if the heart squeezed as a properly used toothpaste tube, not as a collapsing air balloon that collapses upon itself from all directions at the same time, but alas, the heart is far from a good pump. The heart is not as good as it should be at squeezing from back to exit, the intelligent designer was not that intelligent after all, and our invention improves the heart contraction sequence, directing it to go into a properly sequential squeezing.

One of the reasons for the lack of appreciation of this sequential contraction is that it is not perfect, as if it occurred within a well-engineered pump. Moreover, the heart is more or less hanging inside the upper torso, suspended by the blood vessels and somewhat resting on the pericardium, as opposed to a proper peristaltic pump, fixed in relation to the machine in which it works. As a consequence of this, the heart twists and moves on all directions as it pumps, in a dance that masks its sequential motion. Lastly, each half squeezes in ½ second, too short a time for a human being to perceive in detail.

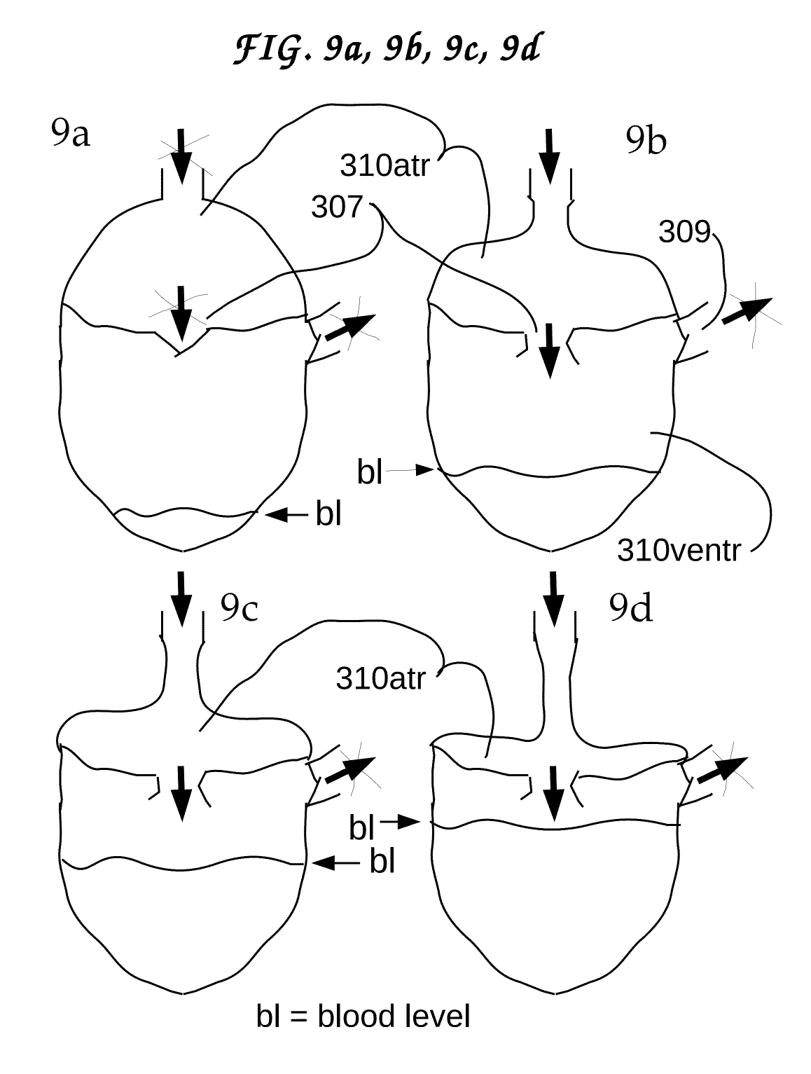

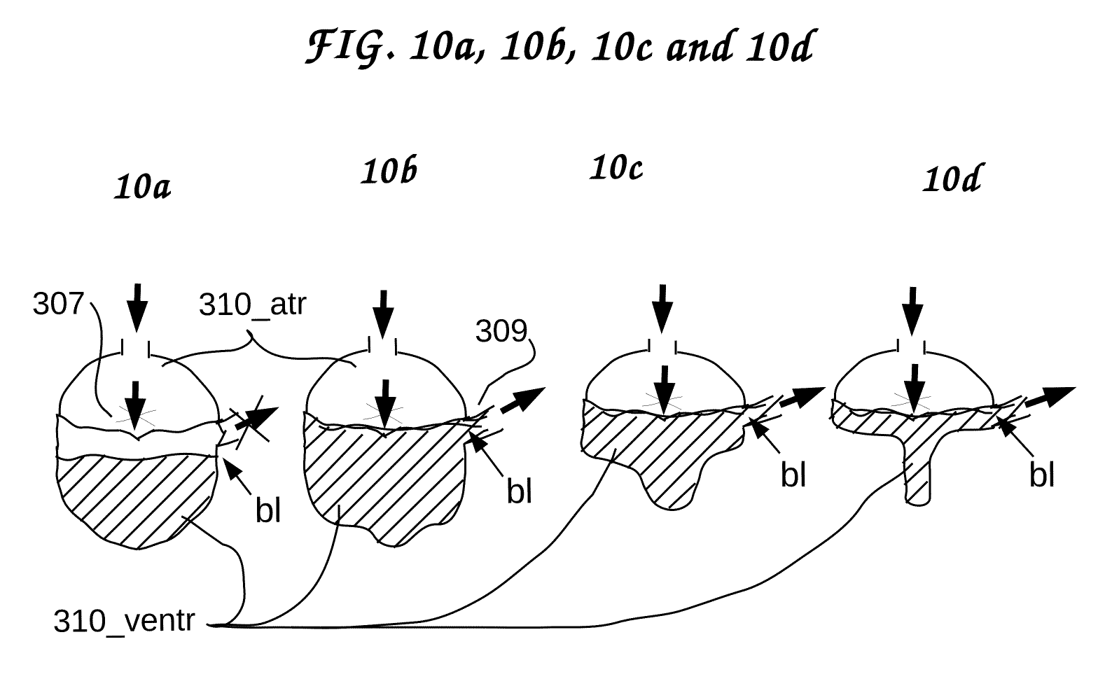

This sequential contraction is valid for all four heart chambers: the right atrium RA, which has its entrance at the top and exit at the bottom, contains the initiating electrical cells at its top (the sino-atrial node, SNA FIG. 1), from which the electrical pulse propagates in its muscle walls from top to bottom, which is, accordingly, the sequential squeezing, as per FIGS. 9a, 9b, 9c and 9d (the figure exaggerates and distorts the situation for display purposes but largely because the inventor is unskilled in drawing and lacks the money to hire a good artist). The ventricle, on the other hand, has both entrance and exit ports at its top, which poses a difficult problem to solve, needing as it does, to contract from bottom to top, to force the blood to exit at the top, while the electric pulse is coming from the top! This was solved by the intelligent designer with a mechanism to arrest the electric pulse at the bottom of the atrium, between the two chambers (else the ventricle would contract from top to bottom, where there is no exit point for the blood!), and another specialized set of cells, the atrium-ventricular node AVN, which, upon receiving the weak electric signal that is coming down from the sino-atrial node SAN, re-start another electric pulse, but with a few milliseconds delay, which is in turn delivered for propagation through a set of specialized fast propagating cells lining the wall between the two ventricles: the short His bundle, followed by the right and left bundles, and finally the Purkinje fibers that spread the electrical pulse throughout the bottom and sides of both ventricles. This trio can be thought as a fast propagating cable which delivers the electric pulse to the bottom of the ventricle or the lower heart chamber. This second electric pulse, delayed from the initial pulse from the sino-atrial node SAN, is then injected at the bottom of the ventricles, from where it propagates upwards, causing an upwards sequential contraction (in the opposite direction as the initial atrium contraction!), as required by an exit point at its top. This process of upwards contraction of the ventricle, the lower chamber, is displayed in FIGS. 10a, 10b, 10c and 10d. This figure displays a cartoon-like version of a well-designed pumping ventricle. During this second stage the one-way tricuspid valve 307 closes, preventing the blood from returning to the upper atrium 310_atr as the lower ventricle 310_ventr contracts from the bottom upwards. At the same time the exit one-way pulmonary valve 309 opens, allowing the blood to flow out of the ventricle 310_ventr. It works, though any respectable intelligent engineer designer would have made a different design, with a ventricular exit at the bottom, not at the top, but at least one can take solace in that this is not the worse design error of the human body—one just has to look at the brain.

The left heart pumping in essentially the same, varying only in minor details, so there is no need to repeat.

This said, the reader should keep in mind two important points here which is the detail on which the whole invention hinges, and which we urge the reader to pay attention and ponder on. Firstly, that not only is the heart contraction caused by an electric pulse but also that this electrical pulse propagates relatively slowly through its muscles and special fibers, because it relies on the propagation of heavy ions in a viscous medium. The propagation of this electrical pulse is very slow as far as electric events happens, the whole process taking just below one second to complete (at a normal heart beating rate of 70 beats per minute). This means that the times involved are of the order of 10 s and even 100 s milliseconds for each part of the cycle, the full cycle taking around 900 milliseconds. This slow propagation time is important for our invention to work, as it will become evident in the sequel. The much faster propagation of electric charges in wires and transistors (1 million times faster), allows that a human-engineered circuit can take over the natural process and improve on it—a very interesting project indeed!

A moment of thought will show the reader that the good operation of the heart depends on the correct propagation of the electric current because the latter determines the former. The electric current propagation in turn depends on the electrical characteristics of the diverse muscles (cells) which comprise the heart, including rapidly electric propagating cells (His fibers, etc), endocardio and miocardio cells, all of which suffer individual variations from person to person, due to their genetic make-up, to which other variations accumulate during the person's lifetime, due to his exercise and eating habits, etc, to which unlucky events as small localized infarctions in the person's later years and heart breaking events in the person's early years, all adding scar tissues with lower conductivity and loss of contraction capability, all adding to a conceptually simple problem, yet of complex analytical solution. This, in turn, is the problem which our invention address: how to better adjust the 3-D electric current propagation through the heart in order to cause the best heart squeezing sequence possible for a particular individual, given his possibilities as determined by the physical conditions of her/his heart.

Another way to say the same thing is to notice that unlike a standard electrical network, on which the paths are discrete and fixed, the electrical path for the current that produces the muscle contraction is continuous over the whole 3-D structure of the heart, and some leak out of it too—these are the pulses measured as EKG signals at the chest surface and even arms and legs surfaces. Because the former, a standard electrical network is composed of discrete, enumerable paths, the information is given as the denumerable branches and nodes, while in the latter case (the heart) the information is a continuous current vector field. For the brain the situation is a little simpler but still it is a continuous path, likewise for other organs, as stomach, bladder, etc.

Besides selecting which electrodes are turned on or off (connected or disconnected from the electrical power), the controlling microprocessor MP1 may, in some incarnations of the device but not necessarily, also select different values of voltages to be connected to the electrodes, both active and passive electrodes. Varying the voltage at the passive electrodes changes the charge deposited on the passive electrodes therefore changing the electric field in its neighborhood, and therefore adjusting the path of the electric current that is injected by the active electrodes. This offers an advantage over currently used heart pacemakers because out invention can better direct the electric current to the particular desirable target volume and avoid entering into undesirable volumes. Also, varying the voltage at the active electrodes, the device can adjust the magnitude of the current that is injected into the heart.

A Graphical User Interface may be used with the invention, which displays the particular device used by the patient (a brain picafina, a heart piquita, a surface planarium or any other), and a series of pull-down menus to select which electrodes are activated and at which electric potential (or current), and any other desired parameter.

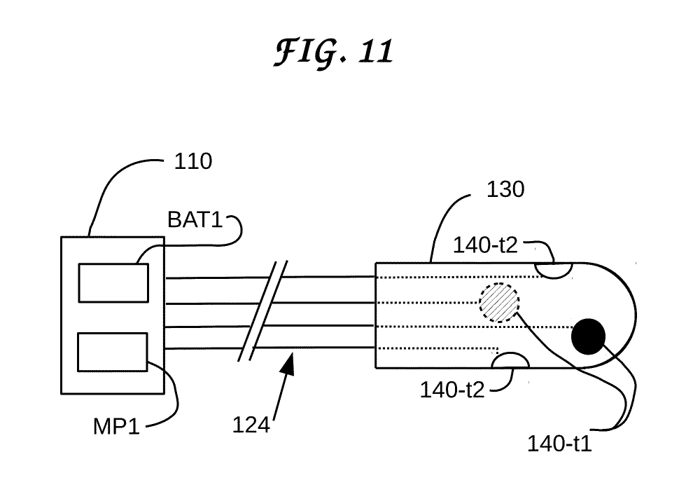

To physically achieve the above description, the controlling mechanism, in this case a microcontroller MC1 residing with the battery/controlling electronics unit BAT1 (FIG. 11) in sealed box 110, is loaded with a computer program (or software), which is capable of executing automatic repetitive tasks following a programmed sequence which offers choices of values to be selected, the details of which are adjusted by a medical professional or by the patient himself, which determines a particular combination of active and passive electrodes to use, including the possibility to able to set the electric potential (voltage) at each electrodes of each type to use, also able to send this information by wires 124 to the stimulating unit 130. Not all these options need to be available in a particular device. For example, a device using the invention could offer only 2 passive and 2 active electrodes and at a single electric potential (voltage) and still be within the scope of the invention. The correct sequence can be determined, for example, by the examination of an EKG (Electro Cardiogram) while varying the active electrodes of each type, their voltages and relative time sequence. Microprocessor MP1, located in box 110, select which wires 124 to be connected to electric power and the voltage level as well, which may be different at each wire 124. Each were 124 connects to one of the electrodes 140-t1 or 140-t2. Each electrode type can be turned on or off (connected or disconnected from the electrical power) under the control of microprocessor MP1.

Theory: The Electric Field Lines.

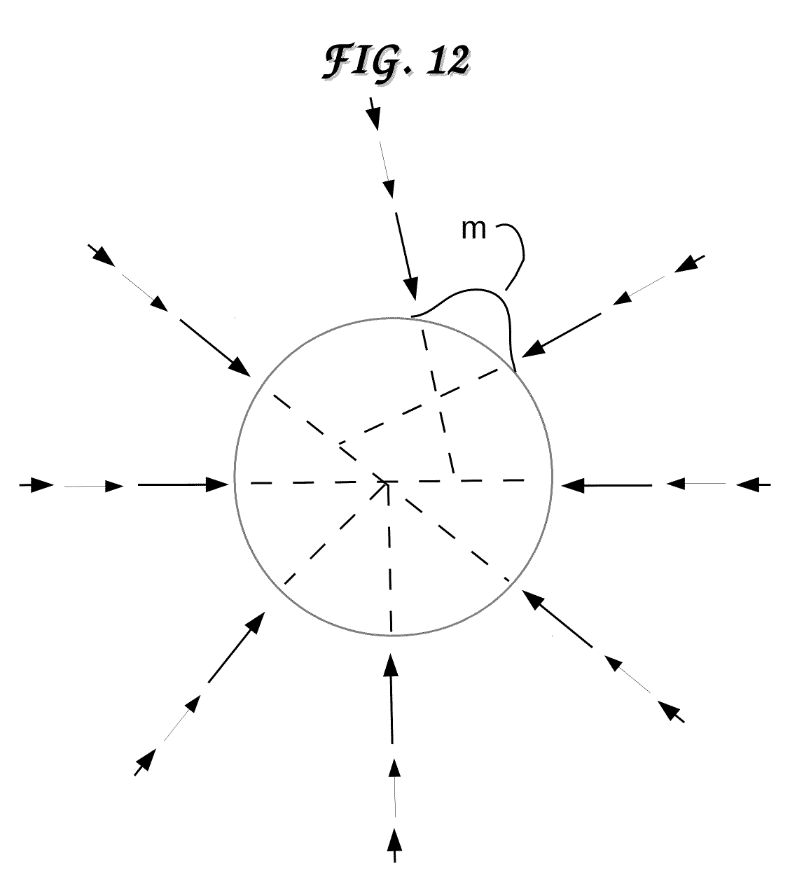

The solution to the problem of the best heart muscle contraction sequence is found in the theoretical analysis of electric current propagation within an electric field. As a side remark, this is similar to the motion of an object by gravity within the gravitational field of the planet, which is vertical towards the center of the planet, assuming a perfectly spherically symmetrical Earth. All objects, unless prevented from falling by some means, do fall down in the direction of the center of the Earth, which defines the straight vertical line. The earth gravitational field is composed of lines radially pointing to its center, as most of the field lines in FIG. 12. FIG. 12 also displays two gravitational field lines next to an exaggerated large mountain, which, due to its large mass tilts the gravitational field lines sideways towards the mountain. An actual large mountain does, surprisingly enough, minutely deflects the gravitational field from its “normal” direction towards the center of the earth, and in amounts that are detectable with modern equipment. This, of course, happens because the mountain attracts sideways.

Given that

F(vector)=q×E(vector),

It follows that the force, and consequently the acceleration and then the motion of an electrically charged particle starting from rest is deflected by the electric field lines. The electric field can take more complex configurations than the gravitational field, because there are two types of electric charges (usually called positive and negative), while the gravitational field is due to only one type of gravitational charge (called mass, they only attract each other). FIGS. 5 (a, b, c, d and e) displays five types of simple electric field configurations: FIG. 5a and FIG. 5b display two cases of field lines that are simpler to calculate, of two electric charges, in fact the configuration normally seen in introductory physics books. The field lines keep deflecting the moving changes towards its direction, the actual path being a combination of the velocity and the deflection caused by the field lines. In other words, the field lines control the flow path of the injected current. From this it follows that to shape the electric field lines is the same as to lay down the “roads” where the current will travel whenever charges are set free in the region. This notion of shaping the field lines to determine the current path is seldom used only because in most electric circuits the current (electric charge) is forced to follow the wires, the coils, the transistors, etc., with no place for an externally imposed electric field to have any effect.

FIG. 5c shows a more complicated case with three charges. The reader is invited to observe the large change of the configuration of the field lines caused by the addition of this third charge, in particular the disappearance of the symmetry that is obvious in figures FIGS. 5a and 5b. FIGS. 5d and 5e display the effect of varying the value of the third charge. Again the reader is invited to ponder on the consequences of varying the values of the charges. Notice that both FIG. 5d and FIG. 5e are asymmetric, yet the shape of the field lines is vastly different between them!

The electric field lines are distinctively unequal, very different shapes. Not displayed is also their strengths, which is also distinct, left out to simplify the figure. FIG. 5 illustrates the point of our invention: a method and a means to conform the electric field lines to the desired 3-D shape required for a most desirable heart squeezing sequence. In fact, using the piquita of our invention, it is possible to even create a 3-D electric field which causes a better heart squeezing sequence than the sequence that happens in a normal, healthy heart, because a normal, typical, healthy heart does not actually follow the best possible sequence! The reader is also asked to observe FIG. 2, which shows another variation of the piquita of our invention with a multiplicity of passive electrodes along the wire that leads to the electrodes. These extra passive electrodes adds to the potential handles with which to shape the electric field lines. Finally, the reader is also asked to remember that each of the passive electrodes may be made of supercapacitors, with the objective of increasing their effect on the field lines, which depend on the total charge at the electrodes, which becomes higher if the electrodes are made as supercapacitors.

Setting each small electrode at the surface of the piquita at a different electric potential (which causes a different electric charge Q on each electrode), a different electric field is set in its neighborhood. The cardiologist, or any other medical personnel, using a computer program to display the electric field created by any particular combination of voltages, will adjust the voltages at different electrodes and see, on the computer screen, the 3-D conformation of the electric field created by them. A Graphical User Interface may be used to enter the choices for voltages, electrodes, etc. Variations of the screen display are still in the scope of the invention. This is one problem of the class known as “inverse problems”, a technical name given in mathematics for problems in which a particular cause is sought (a particular distribution of voltages on the surface of the piquita) which will cause a particular 3-D electric field configuration over the heart muscles. Mathematicians have goose bumps when they are presented with an inverse problem, because they know that most inverse problems have no solution (no closed form solution, to be precise), nor does this one. The solution of such an inverse problem is found by trial and error, adjusting a new charge distribution Q and noticing if the new electric field got closer to the desired one or farther away from it. From this, readjust the charges and observe the result again, and again, etc. Though this may seem a tedious solution, it is easier than working from scratch, because the hearts are approximately the same, and the pacemakers are implanted in approximately the same places, which means that the general type of solution needs to be found once and for all—then only smaller adjustments are necessary. In any case, if so desired the cardiologist can set all the active surface to be at the same electric potential (voltage), and set the passive electrodes at zero voltage, in which case the “improved” electric stimulator (pacemaker) would be working in the same way as prior art pacemakers. In practice, the inventors believe that even without individual adjustments, and only using the best average selection of surface distribution of electric potentials (voltages), there would be some improvement over existing electrical stimulators.

Current devices for heart pacemaking uses two and even three individual electrodes, for example, one electrode near the sino-atrial node (at the top of the right atrium), and one near the bottom of each ventricle (right and left). Multielectrode stimulators much enhance the performance of our invention, because they increase the number of available points over which there is control for adjusting the voltage V (or charge Q, which is the same thing), and also at much larger distances between them. More control is possible with the modern two- and three-stimulators using the technique known as Cardiac Resynchronization Therapy (CRT) than with the one single electrode at the top of the atrium.

Introduction to the Mathematical Treatment of the Problem of the Best Electric Current Distribution Over the Heart Muscle.

It is a well known result in electromagnetic theory that given a volume enclosed by an imaginary closed surface, any arbitrary time-dependent electromagnetic vector field obeying Maxwell's equations can be created adjusting the electric charge distribution at the surface that encloses the closed volume (see Reitz, Milford and Christy (1980), Jackson, (1975) or most any other introductory text in electromagnetic theory). This is valid for electromagnetic waves described by Maxwell's 2^{nd} order differential equations. For the electrostatic case, Dirichlet's principle states that if a scalar function u(x) is a solution to the Poisson's equation

Δu+f=0,

on a domain Ω on Rⁿ (R³ in our case),

with boundary condition u(x)=g(x),

then u(x) can be obtained as the minimizer of the Dirichlet's energy

E{u(x)}=Integral-on-Ω{dx[(½)(grad v)̂2−v*f]}

amongst all twice differentiable functions v(x) such that v(x)=g(x) on the specified domain. In our case u(x) is the electric potential V such that minus grad (V) is the electric field E:

E=−Δ·V

In the medical case, where the volume inside Q. encloses a heart or a brain, etc, so the surface Ω cannot be closed, the above statements are not applicable. Nevertheless, Lara's conjecture for incomplete domains states that a characteristic charge distribution exists on the surface that creates an electric field inside the surface that differs minimally from the desired value for small holes in Ω. Consequently the Lara conjecture guarantees a reasonable solution for the medical case.

Dirichlet's problem is discussed in books dealing with electromagnetism because it is much related to the problems of interest in the field, yet it was initially developed out of its mathematical interest, and it is also discussed in many books in differential equations and potential theory.

This mathematical theory indicates that our invention works better with either a larger area supporting electrodes (which approaches a totally containing surface), as a planarium, and also with just a few small electrodes spread apart, as in the two- and three-electrodes of current heart pacemaking, anchored as they are, at the top of the right atrium and bottom of each ventricle, particularly if a number of passive electrodes are added along the wires leading to the active electrodes at the end of the wires.

### Description and Operation of Alternative Embodiments

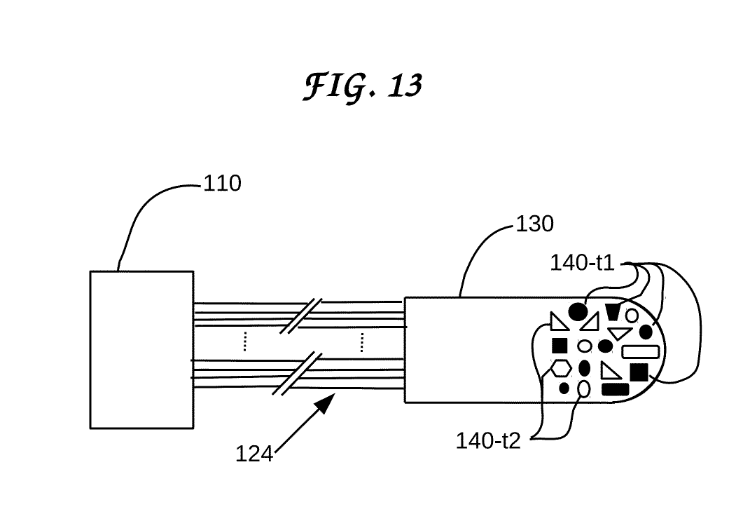

Another embodiment of our invention is application to DBS (Deep Brain Stimulation). In this application the objective is to disrupt the anomalous neurons firings that cause the tremor characteristic of Parkinson's disease, or of what is known as essential tremor. One of the possible solutions is to place an electrode on a chosen target area in the brain then superimpose a current of frequency around 200 Hz on it. FIG. 13 shows a brain-type stimulator we call picafina, similar in structure to stimulators used today, with 4 rings at their distal extremity (Butson and McIntyre (2006)), but with the equivalent electrode described for the heart piquita: passive and active electrodes. The objective for the Deep Brain Stimulator (DBS) of our invention is to adjust the electric field in the vicinity of the picafina brain electric stimulator, to the shape of the particular target volume, which could be the sub-thalamic nucleus (STN), the globus pallidus internus (GPi) or any other. Much effort has been put on the solution of this problem, the solution of which has evaded the practitioners of the art for decades—see, for example, Butson and McIntyre (2006). It can be seen at Butson and McIntyre (2006) that the best solution they proposed is still a symmetric field. Such a symmetric field fail to offer a maximum electrical stimulation in any case, particularly when the electric stimulator happens to have been implanted off-center. As discussed by Butson and McIntyre (2006), this is, in fact, a most common occurrence, due to the small size of the target volumes and their location deep in the base of the brain (for DBS), which is also not directly observed by the surgeon, which inserts the electric stimulator through a one-cm diameter hole drilled at the top of the skull, from where she tries to guide the stimulator tip to the desired target. Our invention allows for more control of the electric field around the stimulator, which in turn, allows for better clinical results. More modern stimulators, e.g. the ones introduced by Sapiens Neuro and by Rubert Martens et al., “Spatial steering of deep brain stimulation volumes using a novel lead design” Clin. Naurophys. Vol 122 pg 558-566 (2011) are capable of creating an asymmetric electric charge distribution in the target area, but fail to decouple the control of the electric field from the injection of the electric charges, therefore failing to maximize the results. Sapiens Neuro brain stimulator is capable of injecting electric charges towards one arc, but not capable of keeping a desired electric field within the target volume to keep the injected charges in the desired volume as our invention does.

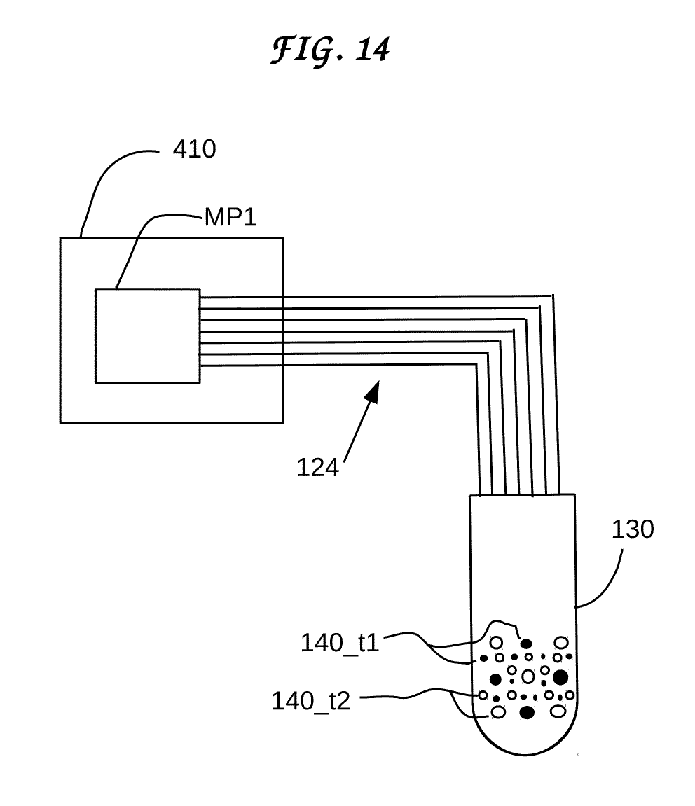

FIG. 14 shows another schematic view of the picafina brain-style stimulator, though other than the stimulator contour, which reminds the DBS stimulating support picafina, the schematic representation could transfer to the heart-type piquita, to the planar type planarium for skin stimulation, and any other. In it, 110 is a hermetically sealed box, which in the existing devices (prior art in legal language) is normally made of titanium or any other bio-compatible material, containing the energy storage unit BAT1 (not shown) and the microprocessor MP1, 124 is the power (voltage or current) wires, one for each electrode, potentially at different voltage/current levels, 130 the picafina stimulator-type, and 140 the plurality of electrodes, some of which are active, others are of the passive type.

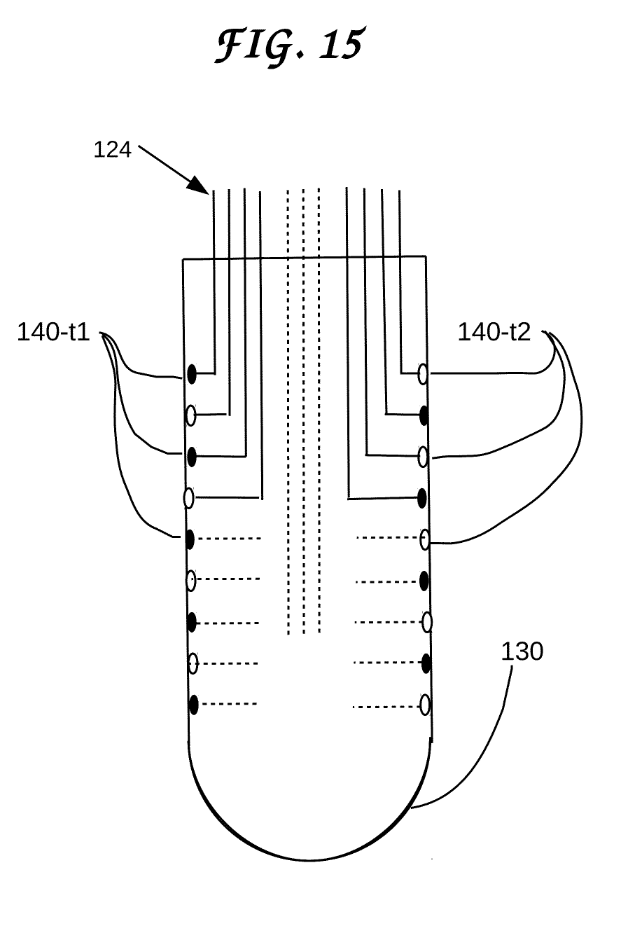

FIG. 15 shows another schematic diagram of a picafina brain-style stimulator of our invention, which, likewise as FIG. 14 reminds the DBS brain picafina but applies equally well to other applications. In this figure the dotted lines indicate more wires, not shown to prevent cluttering of the drawing, one wire for each electrode, and possibly at different voltages/currents. Note also that FIG. 15 omits displaying electrodes on the side of the viewer, for difficulty of making such a drawing, and on the back side, for a similar reason and also for being invisible on the back side. FIG. 15 is a schematic representation, not a real rendition with all details. The same principles are applied to the piquita heart-type stimulator and to other variations of it.

The electrodes for DBS can be of different size, of different shapes and also randomly distributed on the surface of the supporting structure or picafina, or they can be of uniform size and shape, perhaps to decrease manufacturing cost, for example, or to simplify the internal wiring, or any other reason, and they can be also geometrically arranged instead of randomly distributed on the surface. Given the small size of the electrodes, random shape of them is of smaller effect than their numbers, while the use of the two types of electrodes, active or type-1 electrodes and passive or type-2 electrodes are of major importance, given that the latter only change the electric field shape around the stimulator device.

The reader will notice that the DBS application is a natural adaptation of all that is described for the heart pacemaker, yet the DBS is less likely to need time control because there is no sequential muscular contraction, so it is simpler to program and to use than the heart piquita. Yet, situations may arise where time delays between different electrodes may be useful to cause the stimulation to reach some desirable target locations. A multiplicity of electrodes, of variable shapes and sizes, each associated with a unique wire, which is used to select which electrode is turned on, which electrode is turned off, both for type-1 (active) and type-2 (passive). Likewise for the heart pacemaker, the DBS incarnation uses two types of electrodes: a first type, or active type, capable of injecting a current, and a second type, or passive type, which is insulated, not capable of injecting any current (though always there is a small leak current due to insulator imperfections), but which is much useful for creating the vector field around the electrode, which, in turn, determine the 3-D path for the injected current.

Another possible application for the invention is for appetite control. In this application there are at least two possibilities: electrical stimulation on the stomach, and brain stimulation at the locations which are known to control the appetite or at the nerves that carry the information from/to the brain. In the former case the added electrical stimulation may be turned on before a meal, and the electrodes are selected to affect the neurons that send information to the brain regarding the current amount of food in the stomach, which in turn modulate the appetite. If the stimulation is capable to fool the brain, the individual will feel a decreased urge for food, eat less, and lose weight on the long run. This has been used in humans already. The second case, brain stimulation to control the appetite has been only used in animals so far, and with success. For stomach stimulation the shape of the stimulator should be a flat shape to conform to the curvature of the stomach and its enervations, a variation of what we call planarium. For direct brain control it may be similar in shape to the DBS.

Another possible application is for cortical brain stimulation, in which case the stimulator has a flat shape to adjust to the cortical application. We call planarium the flat, or sheet-type stimulator.

Another possible application is for pain control, an improvement of a known device known as TENS (Transcutaneous Electrical Neural Stimulation). In this application the objective is to control superficial pain, as skin pain, and it has used for deeper pain too, as muscle pain. The area in question is in this case surrounded by electrodes attached to the skin, from which a current flows (here it is really an area, the surface area of the skin in question, not what the neurologists call area in the brain, which is a volume). A similar method is in use already, but with larger electrodes, which cannot control the depth and direction of the electric current that is injected, while the passive electrodes of our invention is useful to control the direction and depth of electric current for the same reasons as for the heart muscle, only that in this case the timing is of less importance than with the heart. Also the existing devices use large electrodes, which did not allow for precise control of the point of electric current injection. In this case our invention discloses a large number of small electrodes which are on the surface of the applied patch. Likewise the heart pacemaker, these small electrodes may be numbered or otherwise identifiable by any means, and individually activated by their dedicated wires which is under control of the controlling electronics, are of two types (type-1, or active, and type-2, or passive), and can likewise be turned on at any of a plurality of voltages/currents or off (zero voltage/current). With a wise selection of the active electrodes, it is possible for the medical practitioner to ameliorate the pain felt by the patient in a more effective way than currently used TENS devices because our invention allows for more control of the electric current injected in the patient.

Another variation is the same TENS device described above but with one (or a few) wires used to set a fixed electric potential (voltage), which is then connected to particular electrodes using digital switches or even manual switches. The electrodes may be of either type: active or passive.

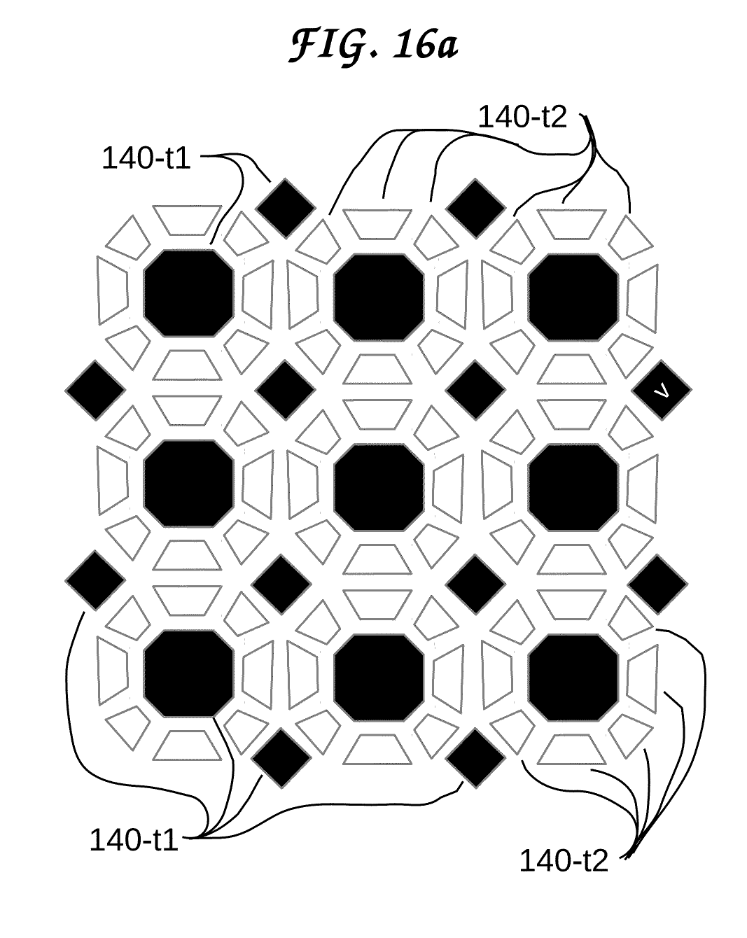

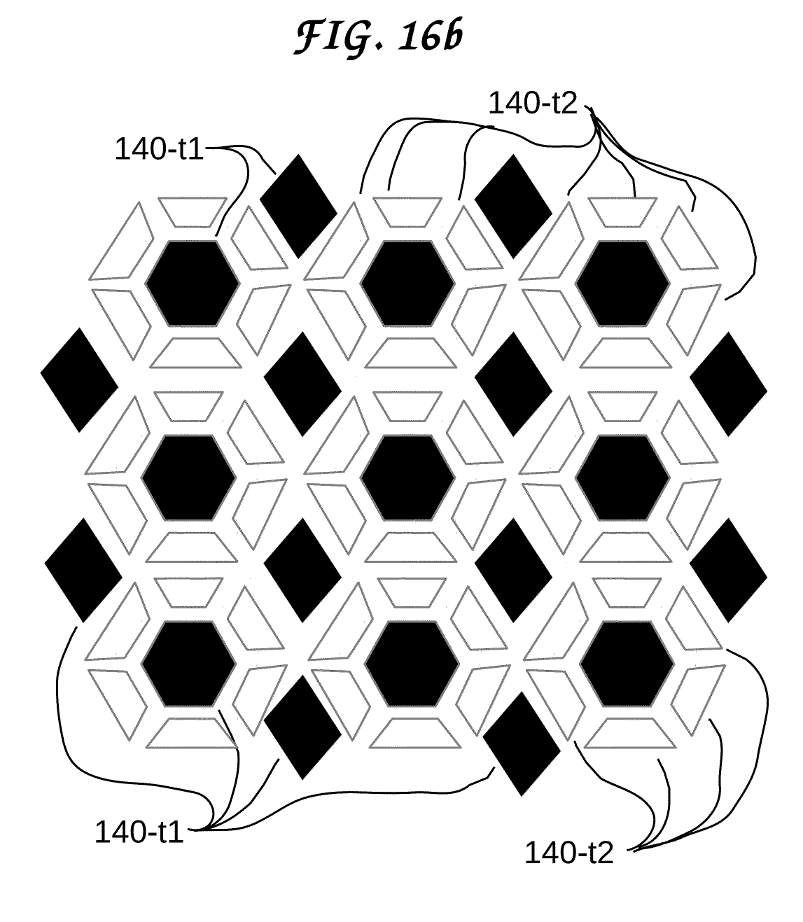

One interesting regular pattern for the electrodes is the hexagonal pattern, which is shown in FIG. 16b, and other variations of it, as the octagonal pattern, shown in FIG. 16a. FIGS. 16a and 16b show two possibilities of the many, with the surrounding electrodes of the active type and the center (hexagonally shaped, octagonal shaped, etc), and the electrode of the passive type surrounding as needed. Other combinations are possible. It is, of course, possible to use only hexagons, because they completely fill a 2-D space. In this case type-1 and type-2 electrodes would alternate, or they could also be random. This particular electrode distribution is symmetrical, which is a departure from the main embodiment, but, given that the electrodes are small, most asymmetric shapes can be approximated. Variations of FIG. 16 are reversing black with white electrodes (that is, reversing active and passive-type), or making them random, each electrode, regardless of their position, center hexagon or one of the surrounding six parallelepid, being assigned randomly to be active or passive. In later use, it is a computer program that determines, from mathematical calculations, which of the electrodes are on and off, in order to create the desired field shape.

Persons familiar with the art understand that the hexagonal pattern displayed at FIG. 16b is just one of the many possibilities. Triangular arrays square arrays, rectangular arrays, and others are possible, these being examples of arrays that completely fill the space. But the individual units do not have to even completely fill the available space, because maximal asymmetry (maximal lack of symmetry, or maximal symmetry breaking) is achieved with random distribution of electrodes of random shapes.

### CONCLUSION, RAMIFICATIONS, AND SCOPE OF INVENTION

The individual electrodes, which in the main embodiment are randomly spread on the supporting structure (piquita), and are of various shapes and sizes, can be all of the same shape and/or same size, and/or can be arranged on an orderly arrangement too. In such a case the advantage of maximal symmetry breaking is not achieved, but some partial symmetry breaking is still obtained with the selection of particular electrodes as the points from which to initiate the stimulation, and the selection of other particular (insulated) electrodes from which to originate the field shaping lines. Cost and other factors could determine a simpler regular electrode arrangement. More orderly arrangements of the electrodes than the arrangement disclosed in the main embodiment, which provides maximal advantage, are still in the scope of the invention. For example, it is possible to control the vector injected electric current (magnitude and direction) with circular electrodes (of either type, active and/or passive ones) that are of different sizes and randomly distributed on the surface of the piquita. It is also possible to control the vector injected electric current with circular electrodes (of either type), that are of the same size and randomly distributed on the surface of the piquita. Or it is also possible to control the injected electric current vector with circular electrodes that are of the same shape and size and orderly distributed on the surface of the piquita, this being the most symmetric electrode arrangement of all. The difference between these options is simply the degree of possible variations and fine control on the vector current, and the choice between each option is based on a cost/benefit analysis, all being still within the scope of our invention.

Persons acquainted with the art of symmetry will recognize that for very small electrodes with small spacing between each, there is little gain if compared with larger electrodes of variable shape and sizes, as particular sets of smaller electrodes can approximately create the shape of a larger electrode of any arbitrary shape. Cost and programming time may dictate one type of another of electrode, and their size and placement, while these variations are still covered in the scope of the invention.

The relative distribution of the electrodes of type-1 and type-2 (current injecting electrodes and electric field shaping electrodes, or magnitude and direction determining electrodes) is random in the main embodiment of this invention, but it is possible to alternate electrodes from type-1 to type-2, then type-1 again, etc., when the electrodes are of the same size and orderly distributed on the surface of the stimulating piquita, picafina, planarium and their variations.

Another way to see the control of the paths of the current in the heart, or the extent of electrical stimulation in brain DBS, etc., is to look at the active electrodes determining the magnitude (and also the direction in a limited way too, because the active electrodes also contribute to the electric field vector though for a very short time) and the passive electrodes determining the direction and speed only of the current injected by the former, active electrodes. In this view one considers the stimulating current as a vector which is directed by the electric field lines.

Other options are possible for the marker 140-tm that indicates the angular position of the piquita with respect to the body in which it is inserted. For example, all the electrodes may have enough X-ray opacity to show in the fluoroscopic images taken during the heart pacemaker implantation. Or one or more or the anchoring arms 131 may be smaller (or larger), or each anchoring arm may be of a different length and/or diameter, to allow their identification.

The main embodiment for heart stimulation uses a simple version of stimulation, which is fixed and continuous, of the type of the old heart pacemakers. It is possible to have stimulation on demand too, as many current pacemakers have, which is based, for example, on activating the stimulation only when the natural pacemaker becomes insufficient, or stops, or becomes erratic. This is called stimulation on demand, easily incorporated in our invention that already contains a microprocessor capable of implementing such decisions. Such extensions are part of the current art of heart pacemakers and may or may not be incorporated in our invention. Our invention is independent of stimulation on demand.

Following lawyer's practice I need to reluctantly add that one skilled in the relevant art, however, will readily recognize that the invention can be practiced without one or more of the specific details, or with other methods, etc. In other instances, well known structures or operations are not shown in detail to avoid obscuring the features of the invention. For example, the details of the wiring can be realized in several different ways, as coiled wires, as printed circuit wires, etc., many or most of which are compatible with the invention, and therefore the details of these, and other details are not included in this patent disclosure.

### SEQUENCE LISTING

Not applicable

### APPENDIX

### Drawings

### List of Reference Numerals

BAT1=Battery inside sealed box, usually implanted in the patient's chest.
MP1=Microprocessor 1. One of the possible units capable of executing a programmable sequence of instructions, as the venerable 8085, or the 8086 (which was the brain of the first IBM-PC), 80286, 80386, 80487, pentium, DSP, microcontrollers, etc. Some of these may include memory, DAC, ADC, and interface devices.
100=body of picafina of our invention.
110=sealed box containing the electrical energy storage unit (e.g., a battery) and the microprocessor MP1.
124=power conveying means. This may be printed circuit wires but may be standard wires or other power conveying means.
130=ST1=electrical stimulating probe, in the main embodiment is fixed in the inner part of the heart, brain, or other organs.
131=anchoring arms to prevent the heart stimulator type (piquita) from moving back once it is forced into the endocardio/miocardio, also known as “tines”
132=main body of piquita heart pacemaker.
140_{—}t1=type1 or active electrodes (standard electrodes, capable of injecting current in its neighborhood).
140_{—}t2=type2 or passive electrodes (electrically insulated electrodes, capable of influencing the electric field lines, but not capable to inject current). Typically type 2, passive electrodes are covered by a silicon dioxide layer, but any other insulator is possible, the type of insulator being not important for our invention.
140_{—}tm=opaque marker (for X-rays, or for MRI, or for ultrasound, etc), to indicate position of the implanted device in the patient's body.
307=tricuspid valve, between the right atrium and ventricle.
309=pulmonary valve, exit from the right ventricle.
310_atr=atrium.
310_ventr=ventricle.
320=layer inside the stimulator 132 showing electrical connections to electrodes and connector at the top to connect to power conveying wires 124.
330, 340, . . . =same layers as 320.

### Alphabetical Labels

AVN=Atrial-ventricular node.
bl=blood level.
C1=cable 1
C2=cable 2
HB=His Bundle.
LBB=Left bundle branch.
LA=Left atrium.
LV=Left ventricle.
m=mountain (exaggerated height for display)
PF=Purkinje fibers.
RA=right atrium.
RBB=Right bundle brunch.
RV=Right ventricle.
SC1_1=Supercapacitor 1 at cable1
SC2_1=supercapacitor 2 at cable1
SCV=Subclavian vein
SNA=sino-atrial node.
ST1=Stimulator, same as 132

### REFERENCES

Butson and McIntyre (2006). Christopher R. Butson and Cameron C. McIntyre “Role of electrode design on the volume of tissue activated during deep brain stimulation” Journal of Neural Engineering, vol. 3, pgs. 1-8 (2006)
Chong Il Lee and Sergio Lara Pereira Monteiro (2011) “Method and means to address and make use of multiple electrodes for measurements and electrical stimulation in neurons and other cells including brain and heart” US patent application Ser. No. 13/053,137, Mar. 21, 2011, not yet published.
Chong Il Lee (2010) “Method and means for connecting a large number of electrodes to a measuring device” US patent application number 20100079156, published Apr. 1, 2010
Chong Il Lee and Sergio Lara Pereira Monteiro (2010) “Method and means for connecting and controlling a large number of contacts for electrical cell stimulation in living organisms” U.S. patent application number 20100082076, published Apr. 1, 2010.
Colleen Clancy and Yang Xiang “Wrapped around the heart” News and Views, Nature 507, 43, (6 Mar. 2014), attached.
DIRICHLET—http://en.wikipedia.org/wiki/Dirichlet_principle
JamilleHetke_Kipke_Pellinen_Anderson_ModularMultichannelMicroelectodeArrayEtc_USPTO-PatPubl-US2007-0123765_{—}070531
Jackson (1975) Jackson “Classical Electrodynamics” Wiley.
Lizhi Xu et al. “3D multifunctional integumentary membranes for spatiotemporal cardiac measurements and stimulation across the entire epicardium”, Nature Communications DOI:10.1038/ncomms4329, attached.
Medtronic (n/d) Medtronic website with info on DBS leads.
http://professional.medtronic.com/pt/neuro/dbs-md/prod/dbs-lead-model-3387/index.htm http://professiona.medtronic.com/pt/neuro/dbs-pd/prod/dbs-lead-model-3391/index.htm
3) Pierre Martin “Une membrane artificielle pour surveiller le coeur” La Recherche no. 487 page 20, 1o Mai 2014, attached.
[Plachta 2014] Dennis T. T. Plachta et al. “Blood pressure control with selective vagal nerve stimulation and minimal side effects” J. Neural Eng. 11, 036011 (2014).
Reitz, Milford & Christy (1980), John Reitz, Frederick Milford, Robert Christy “Foundations of Electromagnetic Theory” 3^{rd} edition, 1980.
Thaler (2003) Malcolm S. Thaler “The Only EKG Book You'll Ever Need”, Lippincott Williams & Wildins, 4^{th} ed. (2003).

1. An electrical stimulating device comprising:
an electric energy storage unit,
a controlling electronics,
a supporting structure comprising a proximal extremity, a distal extremity, an inner lumen with an outer surface;
a plurality of electrodes comprising at least one electrode belonging to a group of passive electrodes;
a plurality of required means to electrically interconnect these parts;
wherein the passive electrodes are configured to project electric fields into the body cells surrounding the supporting structure while configured not to inject electric currents into the body cells surrounding the supporting structure;
wherein the controlling electronics comprises electronic circuits to select a subset of the plurality of electrodes to be operational;
wherein an electrically insulating layer on the passive electrodes act as a insulator for DC current or for low frequencies signals, as opposed to the insulating layer to create a capacitor for capacitive coupling of high frequency AC current;
wherein the passive electrodes are configured to create an electric vector field in the body cells surrounding the supporting structure, the electric vector field characterized by a magnitude and a direction, wherein the direction determines a plurality of field lines, which redirects any existing electric current;
wherein the electric field lines projected by the passive electrodes direct the path of moving electric charges in the body cells where the electric field lines are located.

2. The stimulating device of claim 1 where the at least some of the plurality of electrodes belonging to a group of passive electrodes is made as a supercapacitor.

3. The stimulating device of claim 1 where the plurality of required means to electrically interconnect these parts are made with printed circuit technology.

4. The device of claim 1 with an extra radio receiver or a similar action-at-a-distance communication device capable of receiving instructions from an external unit and passing the instructions to the controlling electronics,

5. The device of claim 1 designed for use in a heart.

6. The device of claim 1 designed for use in a brain.

7. A multi electrode electric stimulating method comprising:
providing a set of type-1 active electrodes at a first location;
providing a set of type-2 passive electrodes a first location;
providing an electric energy storage device and a controlling electronics at a second location;
providing dedicated wires connecting each of the type-1 and type-2 electrodes to the controlling electronics and the energy storage device;
wherein the type-1 electrodes are capable of injecting electric currents in the cells surrounding itself;
wherein the type-2 electrodes are supercapacitors covered by an insulating layer that prevents current from flowing out of their surfaces into the surrounding cells;
wherein the connecting wires can be connected to a multiplicity of voltage and/or current levels.

8. A computer program for controlling a system for adjusting an electric field in a region of space, comprising program code means for selecting unique electric potential values for each of a plurality of supercapacitors;
wherein the unique electric potential values cause a unique charge values on each supercapacitor;
wherein each of the charges causes a unique contribution for the total value of the electric field in the three dimensional region of space.
